(19) United States
(12) Patent Application Publication
(10) Pub. No.: US 2026/0232625 A1
(43) Pub. Date: Aug. 13, 2026

(54) NOVEL DECURSIN DERIVATIVE AND USE THEREOF FOR PREVENTING OR TREATING NON-SMALL CELL LUNG CANCER

(71) Applicant: JAEIN R&P INC., Seoul (KR)

(72) Inventors: Seong-Gyu KO, Seoul (KR); Jae Yeol LEE, Seoul (KR); Junseong AHN, Daejeon (KR); Hyemin CHOI, Seoul (KR); Kangwook LEE, Seoul (KR); Hyun-Ha HWANG, Seoul (KR); Minju GWON, Seoul (KR); Minji KIM, Jeonju-si (KR)

(73) Assignee: JAEIN R&P INC., Seoul (KR)

(21) Appl. No.: 19/157,446

(22) PCT Filed: Feb. 6, 2024
(86) PCT No.: PCT/KR2024/095083

§ 371 Date: Aug. 18, 2025

(30) Foreign Application Priority Data
Feb. 16, 2023 (KR) 10-2023-0020679

(51) Int. Cl. A61K 31/353; A61K 31/4178; A61K 31/4433; A61P 35/00; C07D 493/04

(52) U.S. Cl. CPC A61K 31/353; A61K 31/4178; A61K 31/4433; A61P 35/00; C07D 493/04

(57) ABSTRACT

The present disclosure relates to a series of novel decursin derivatives, a method for preparing the same, and use thereof for preventing or treating non-small cell lung cancer.

### TECHNICAL FIELD

The present disclosure relates to a series of novel decursin derivatives, a method for preparing the same, and use thereof for preventing or treating non-small cell lung cancer.

### BACKGROUND ART

Lung cancer, which is a malignant tumor originating from the lung, is largely classified into small cell lung cancer (SCLC) and non-small cell lung cancer (NSCLC) based on the histological type. Although SCLC is classified as a part of lung cancer according to the location of the tissue of onset, it is distinguished from other lung cancers in terms of clinical course, therapeutic methods, and prognosis, and is therefore classified separately as described above. In addition, NSCLC is classified into adenocarcinoma, squamous cell carcinoma, and large-cell carcinoma according to histological type.

Among them, NSCLC is classified into adenocarcinoma, squamous cell carcinoma, and large-cell carcinoma. First, adenocarcinoma mainly occurs in the peripheral region of the lung and occurs frequently even in women or non-smokers. It is often accompanied by metastasis even when small in size, and has recently shown an increasing incidence. Next, squamous cell carcinoma is mainly found in the central region of the lung and grows primarily into the bronchial lumen causing obstruction of the bronchi. It is common in men and is known to be closely associated with smoking. Lastly, large-cell carcinoma mainly occurs near the lung surface (lung periphery), about half of cases occur in the large bronchi, and accounts for about 4 to 10% of all lung cancers. It generally has a large cell size, and some of them show rapid proliferation and metastasis, and is known to have a worse prognosis compared to other NSCLCs. Meanwhile, NSCLC exhibits a low survival rate due to a very low diagnostic success rate, and the survival rate over the past ten years has been only about 10%.

NSCLC treatment includes cytotoxic chemotherapy using platinum-based doublets, EGFR-targeted therapy using EGFR inhibitors and anti-EGFR monoclonal antibodies, angiogenesis inhibitors using anti-VEGF monoclonal antibodies and VEGFR2 antagonists, and immunotherapies using anti-CTLA-4 monoclonal antibodies and anti-PD-1 monoclonal antibodies. Each of the above therapeutic methods has its own advantages, but also has limitations, and therefore, it is necessary to develop new therapeutic agents with reduced side effects to overcome such limitations.

### DISCLOSURE

### Technical Problem

One object of the present invention is to provide a series of novel decursin derivative compounds, a tautomer thereof, a stereoisomer or a mixture thereof, or a pharmaceutically acceptable salt thereof.

Another object of the present invention is to provide a method for preparing the novel decursin derivative compounds, a tautomer thereof, a stereoisomer or a mixture thereof, or a pharmaceutically acceptable salt thereof.

Still another object of the present invention is to provide a pharmaceutical composition for preventing or treating non-small cell lung cancer (NSCLC), comprising, as an active ingredient, the decursin derivative compounds, a tautomer thereof, a stereoisomer or a mixture thereof, or a pharmaceutically acceptable salt thereof.

Still another object of the present invention is to provide a composition for combination therapy for preventing or treating NSCLC, comprising, as active ingredients, the decursin derivative compounds, a tautomer thereof, a stereoisomer or a mixture thereof, or a pharmaceutically acceptable salt thereof, and gefitinib.

Still another object of the present invention is to provide a method for treating non-small cell lung cancer (NSCLC), comprising administering the composition for combination therapy to a subject in need.

### Advantageous Effects

The series of novel decursin derivative compounds according to the present invention can provide a solution to the correlation between compound structure and pharmacological activity. In addition, a specific derivative compound, (S,E)-2,2-dimethyl-8-oxo-2,3,4,8-tetrahydropyrano[3,2-g]chromen-3-yl 3-(furan-3-yl)acrylate, which exhibits a killing effect on non-small cell lung cancer cell lines screened therefrom, exhibits a superior effect compared to decursin, which is originally known to have anticancer activity, and can further exhibit a synergistic effect when administered in combination with gefitinib, a non-small cell lung cancer treatment agent.

### BRIEF DESCRIPTION OF THE DRAWINGS

FIGS. 1a to 1d are diagrams showing cytotoxicity against A549 according to the concentration of compounds 1 to 30 according to an embodiment of the present invention. (a) to (c) show the results by method 1, and (d) shows the results by method 2.

FIG. 2 is a diagram showing cytotoxicity against A549 following co-administration of gefitinib and decursin or a decursin derivative compound of Example 5.

FIG. 3 is a diagram showing the synergy score for A549 cell death according to co-administration of gefitinib and decursin or a decursin derivative compound of Example 5.

### DETAILED DESCRIPTION OF PREFERRED EMBODIMENTS

Each description and embodiment described herein may be applied to other descriptions and embodiments, respectively. That is, all combinations of various elements described herein fall within the scope of the present invention. Further, the scope of the present invention is not limited by the specific description described below.

Furthermore, those skilled in the art may recognize or identify numerous equivalents to the specific embodiments of the present invention described herein by using routine experimentation. Moreover, such equivalents are intended to be included within the present invention.

In addition, throughout the specification of the present invention, when it is stated that a part “comprises” a certain component, it means that the part may further include other components, rather than excluding all other components, unless explicitly stated otherwise.

Hereinafter, the present invention will be described in more detail.

In order to achieve the foregoing objects, the first aspect of the present invention provides a compound represented by Formula 1 below, a tautomer thereof, a stereoisomer or a mixture thereof, or a pharmaceutically acceptable salt thereof:

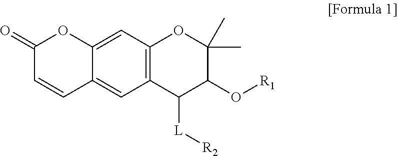

wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

For example, in Formula 1 above, when L is a single bond, R₂ may be hydrogen, and when L is —NH—, R₁ may be hydrogen, but is not limited thereto.

For example, in Formula 1 above, any one of R₁ and R₂ may be hydrogen, and the other may be C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl, but is not limited thereto.

For example, in Formula 1 above, any one of R₁ and R₂ may be hydrogen and the other may be 1-methyl-2-phenylethenylcarbonyl, 2-phenylethenylcarbonyl, trifluoromethyl-substituted 2-phenylethenylcarbonyl, dimethylaminosulfonyl-substituted 2-phenylethenylcarbonyl, dimethylamino-substituted 2-phenylethenylcarbonyl, phenylmethylcarbonyl, dimethylamino-substituted phenylmethylcarbonyl, (pyridinylethyl)carbonyl, (benzo[d][1,3]dioxolylethyl)carbonyl, (imidazolylethyl)carbonyl, (furanylethyl)carbonyl, (pyridinylethenyl)carbonyl, (benzo[d][1,3]dioxolylethenyl)carbonyl, (imidazolylethenyl)carbonyl, or (furanylethenyl)carbonyl, but is not limited thereto.

More specifically, the compound may be:
1. (S)-2,2-dimethyl-8-oxo-2,3,4,8-tetrahydropyrano[3,2-g]chromen-3-yl 3-(1H-imidazol-1-yl)propanoate,
2. (S)-2,2-dimethyl-8-oxo-2,3,4,8-tetrahydropyrano[3,2-g]chromen-3-yl 3-(pyridin-2-yl)propanoate,
3. (S,E)-2,2-dimethyl-8-oxo-2,3,4,8-tetrahydropyrano[3,2-g]chromen-3-yl 3-(pyridin-2-yl)acrylate,
4. (S,E)-2,2-dimethyl-8-oxo-2,3,4,8-tetrahydropyrano[3,2-g]chromen-3-yl 2-methyl-3-phenylacrylate,
5. (S,E)-2,2-dimethyl-8-oxo-2,3,4,8-tetrahydropyrano[3,2-g]chromen-3-yl 3-(furan-3-yl)acrylate,
6. (S)-2,2-dimethyl-8-oxo-2,3,4,8-tetrahydropyrano[3,2-g]chromen-3-yl 3-(benzo[d][1,3]dioxol-5-yl)propanoate,
7. (S,E)-2,2-dimethyl-8-oxo-2,3,4,8-tetrahydropyrano[3,2-g]chromen-3-yl 3-(benzo[d][1,3]dioxol-5-yl)acrylate,
8. (S,E)-2,2-dimethyl-8-oxo-2,3,4,8-tetrahydropyrano[3,2-g]chromen-3-yl 3-(4-(N,N-dimethylsulfamoyl)phenyl)acrylate,
9. (S,E)-2,2-dimethyl-8-oxo-2,3,4,8-tetrahydropyrano[3,2-g]chromen-3-yl 3-(4-(trifluoromethyl)phenyl)acrylate,
10. (R)-2,2-dimethyl-8-oxo-2,3,4,8-tetrahydropyrano[3,2-g]chromen-3-yl 3-(1H-imidazol-1-yl)propanoate,
11. (R)-2,2-dimethyl-8-oxo-2,3,4,8-tetrahydropyrano[3,2-g]chromen-3-yl 3-(pyridin-2-yl)propanoate,
12. (R,E)-2,2-dimethyl-8-oxo-2,3,4,8-tetrahydropyrano[3,2-g]chromen-3-yl 3-(pyridin-2-yl)acrylate,
13. (R,E)-2,2-dimethyl-8-oxo-2,3,4,8-tetrahydropyrano[3,2-g]chromen-3-yl 2-methyl-3-phenylacrylate,
14. (R,E)-2,2-dimethyl-8-oxo-2,3,4,8-tetrahydropyrano[3,2-g]chromen-3-yl 3-(furan-3-yl)acrylate,
15. (R)-2,2-dimethyl-8-oxo-2,3,4,8-tetrahydropyrano[3,2-g]chromen-3-yl 3-(benzo[d][1,3]dioxol-5-yl)propanoate,
16. (R,E)-2,2-dimethyl-8-oxo-2,3,4,8-tetrahydropyrano[3,2-g]chromen-3-yl 3-(benzo[d][1,3]dioxol-5-yl)acrylate,
17. (R,E)-2,2-dimethyl-8-oxo-2,3,4,8-tetrahydropyrano[3,2-g]chromen-3-yl 3-(4-(N,N-dimethylsulfamoyl)phenyl)acrylate,
18. (R,E)-2,2-dimethyl-8-oxo-2,3,4,8-tetrahydropyrano[3,2-g]chromen-3-yl 3-(4-(trifluoromethyl)phenyl)acrylate,
19. N-((3S,4R)-3-hydroxy-2,2-dimethyl-8-oxo-2,3,4,8-tetrahydropyrano[3,2-g]chromen-4-yl)-3-(1H-imidazol-1-yl)propanamide,
20. N-((3S,4R)-3-hydroxy-2,2-dimethyl-8-oxo-2,3,4,8-tetrahydropyrano[3,2-g]chromen-4-yl)-3-(pyridin-2-yl)propanamide,
21. (E)-N-((3S,4R)-3-hydroxy-2,2-dimethyl-8-oxo-2,3,4,8-tetrahydropyrano[3,2-g]chromen-4-yl)-3-(pyridin-2-yl)acrylamide,
22. (E)-N-((3S,4R)-3-hydroxy-2,2-dimethyl-8-oxo-2,3,4,8-tetrahydropyrano[3,2-g]chromen-4-yl)-2-methyl-3-phenylacrylamide,
23. (E)-3-(furan-3-yl)-N-((3S,4R)-3-hydroxy-2,2-dimethyl-8-oxo-2,3,4,8-tetrahydropyrano[3,2-g]chromen-4-yl)acrylamide,
24. 3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)propanamide,
25. (E)-3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)acrylamide,
26. (E)-3-(4-(N,N-dimethylsulfamoyl)phenyl)-N-((3S,4R)-3-hydroxy-2,2-dimethyl-8-oxo-2,3,4,8-tetrahydropyrano[3,2-g]chromen-4-yl)acrylamide,
27. (E)-N-((3S,4R)-3-hydroxy-2,2-dimethyl-8-oxo-2,3,4,8-tetrahydropyrano[3,2-g]chromen-4-yl)-3-(4-(trifluoromethyl)phenyl)acrylamide,
28. (S)-2,2-dimethyl-8-oxo-2,3,4,8-tetrahydropyrano[3,2-g]chromen-3-yl 3-(furan-3-yl)propanoate,
29. (S)-2,2-dimethyl-8-oxo-2,3,4,8-tetrahydropyrano[3,2-g]chromen-3-yl 4-(dimethylamino)phenylacetate, or
30. (S,E)-2,2-dimethyl-8-oxo-2,3,4,8-tetrahydropyrano[3,2-g]chromen-3-yl 3-(4-(dimethylamino)phenyl)acrylate, but is not limited thereto.

For example, the compound of the present invention may exist in a form of a pharmaceutically acceptable salt. Useful salts include acid salts formed by pharmaceutically acceptable free acids. As used herein, the term “pharmaceutically acceptable salt” refers to a form of salt that is relatively non-toxic and harmless at an effective concentration to a patient and refers to any organic or inorganic addition salt of the compound represented by Formula 1 in which the side effects of the salt do not reduce the beneficial effects of the compound.

Acid addition salts are prepared by conventional methods, for example, by dissolving the compound in an excess of aqueous acid solution and precipitating the salt using a water-miscible organic solvent, such as methanol, ethanol, acetone, or acetonitrile. The compound, and an acid in water or alcohol (for example, glycol monomethyl ether) in equal molar amounts are heated, and subsequently, the mixture may be evaporated to dryness, or the precipitated salt may be suction filtered.

In particular, as the free acids, organic acids and inorganic acids may be used; inorganic acids such as hydrochloric acid, phosphoric acid, sulfuric acid, nitric acid, and tartaric acid may be used, and organic acids such as methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid may be used, but the free acids are not limited thereto.

In addition, a pharmaceutically acceptable metal salt may be prepared using a base. An alkali metal salt or alkaline earth metal salt is obtained, for example, by dissolving the compound in an excess of an alkali metal hydroxide or alkaline earth metal hydroxide solution, filtering out the undissolved compound salt, and evaporating and drying the filtrate. In particular, as the metal salts, it is pharmaceutically suitable to prepare sodium, potassium, or calcium salts, but the metal salts are not limited thereto. Also, a corresponding silver salt may be obtained by reacting an alkali metal or alkaline earth metal salt with a suitable silver salt (for example, silver nitrate).

The pharmaceutically acceptable salt of the compound of the present invention, unless otherwise indicated, includes salts of acidic or basic groups that may be present in the compound of Formula 1. For example, pharmaceutically acceptable salts may include sodium, calcium, and potassium salts of the hydroxy group, and other pharmaceutically acceptable salts of the amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts; these salts may be prepared by salt preparation methods known in the art.

Any pharmaceutically acceptable salt of decursing derivative compound may be used as the salt of the decursin derivative compound of the present invention without limitation, as long as the salt is pharmaceutically acceptable and exhibits pharmacological activity equivalent to that of the decursin derivative compound.

In addition, the compound represented by Formula 1 according to the present invention includes not only pharmaceutically acceptable salts thereof, but also solvates such as hydrates that can be prepared therefrom, and all possible stereoisomers without limitation. The solvates and stereoisomers of the compound represented by Formula 1 may be prepared from the compound represented by Formula 1 using a method known in the art.

Furthermore, the compound represented by Formula 1 according to the present invention may be prepared in a crystalline or amorphous form, and if prepared in a crystalline form, the compound may be optionally hydrated or solvated. In the present invention, compounds containing various amounts of water may be included in addition to the stoichiometric hydrate of the compound represented by Formula 1. The solvates of the compound represented by Formula 1 according to the present invention include both the stoichiometric solvates and non-stoichiometric solvates.

The second aspect of the present invention provides a method for preparing a compound represented by Formula 1 below, a tautomer thereof, a stereoisomer thereof or a mixture thereof, or a pharmaceutically acceptable salt thereof, comprising:
a step (1-1) of reacting (S)-7-hydroxy-8,8-dimethyl-7,8-dihydropyrano[3,2-g]chromen-2(6H)-one (decursinol), or (R)-7-hydroxy-8,8-dimethyl-7,8-dihydropyrano[3,2-g]chromen-2(6H)-one with R1-OH in the presence of 4-(dimethylamino)pyridine (DMAP) and 1-ethyl-3-(3-dimethylaminopropyl)carbodiimide hydrochloride (EDC-HCl), or
a step (1-2) of reacting (6R,7S)-6-amino-7-hydroxy-8,8-dimethyl-7,8-dihydropyrano[3,2-g]chromen-2(6H)-one with R₂—OH in the presence of 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium; HATU) and N,N-diisopropylethylamine (DIPEA):

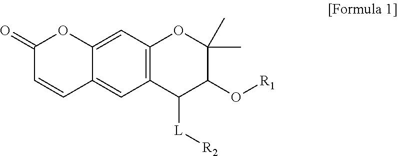

wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

For example, the step (1-1) may be performed by Steglich esterification. Specifically, the step (1-1) is performed for 30 minutes to 48 hours by heating to 10° C. to 40° C. following dissolving the reactants in an organic solvent at −5° C. to 10° C., but is not limited thereto. The organic solvent may be dichloromethane (DCM), but is not limited thereto.

For example, the step (1-2) may be performed by amide coupling. Specifically, the step (1-2) may be performed in an organic solvent for 30 minutes to 48 hours, but is not limited thereto. The organic solvent may be tetrahydrofuran (THF), but is not limited thereto.

For example, the (S)-7-hydroxy-8,8-dimethyl-7,8-dihydropyrano[3,2-g]chromen-2(6H)-one may be prepared by separation from an Angelica gigas extract.

Specifically, the root extract of Angelica gigas Nakai was extracted with a lower alcohol, such as ethanol, at 60 to 90° C., fractionated with water and ether, and the ether layer was collected. Then, a mixed solution of water and THF and an excess amount of NaOH were added to the extract, followed by hydrolysis, and the extract was adjusted to acidity by adding a concentrated aqueous hydrochloric acid solution, followed by concentration. Thereafter, water and dichloromethane (DCM) may be added, and the dichloromethane may be fractionated to obtain the DCM layer, but the method is not limited thereto.

For example, the (R)-7-hydroxy-8,8-dimethyl-7,8-dihydropyrano[3,2-g]chromen-2(6H)-one may be prepared by:
a step (a-1) of reacting, in an organic solvent, (S)-7-hydroxy-8,8-dimethyl-7,8-dihydropyrano[3,2-g]chromen-2(6H)-one with triphenylphosphine at 45° C. to 80° C. for 30 minutes to 5 hours;
a step (a-2) of adding a compound prepared according to the step (a-1) and (R,R)-Jacobsen's catalyst to a mixed solution of NaOCl and NaHPO₄ adjusted to pH 10 to 12, and reacting at −5° C. to 10° C. for 24 to 100 hours; and
a step (a-3) of dissolving a compound prepared according to the step (a-2) and NaCNBH₃ in an organic solvent, cooling to −5° C. to 10° C., and reacting by adding BF₄OEt₂, but is not limited thereto.

For example, the (6R,7S)-6-amino-7-hydroxy-8,8-dimethyl-7,8-dihydropyrano[3,2-g]chromen-2(6H)-one may be prepared by:
a step (b-1) of reacting, in an organic solvent, (S)-7-hydroxy-8,8-dimethyl-7,8-dihydropyrano[3,2-g]chromen-2(6H)-one with triphenylphosphine at 40° C. to 80° C. for 30 minutes to 5 hours;
a step (b-2) of Jacobsen epoxidation, namely adding a compound prepared according to the step (b-1) and (S,S)-Jacobsen's catalyst to a mixed solution of NaOCl and NaHPO₄ adjusted to pH 10 to 12, and reacting at −5° C. to 10° C. for 24 to 100 hours; and
a step (b-3) of reacting a compound prepared according to the step (b-2), in a lower alcohol solvent, with ammonia for 3 to 24 hours, but is not limited thereto.

For example, the steps (a-1) and (b-1) may be performed by Appel reaction, but are not limited thereto. The organic solvent may be a mixed solvent of acetonitrile (ACN) and carbon tetrachloride (CCl₄), for example, a mixed solvent in a volume ratio of 1:1, but is not limited thereto.

For example, the steps (a-2) and (b-2) may be performed by Jacobsen epoxidation, but are not limited thereto. Specifically, the step (a-2) may be performed by preparing a solution in which 33 mL of 15% NaOCl and 13 mL of 0.05 M NaHPO₄ are mixed and the pH is adjusted by adding 1 M NaOH or 1 M HCl, and then adding the prepared solution to a solution in which the compound prepared according to step (b-1) and the (S,S)-Jacobsen catalyst are dissolved in DCM, but is not limited thereto.

For example, the step (a-3) may be performed in THF, but is not limited thereto.

For example, the step (b-3) may be performed in MeOH, but is not limited thereto.

For example, in the method of the present invention, the reactants and/or precursors used in each step may be purchased as commercially available compounds, or the reactants or intermediates of each step may be purchased to synthesize the compounds through a single reaction known in the art or a combination of several reactions, but are not limited thereto.

In addition, after each reaction, a process of separating and/or purifying the product may be further comprised as necessary, which may be carried out using various methods known in the art. For example, the process may be carried out by extraction, trituration, and/or column chromatography, but is not limited thereto.

The third aspect of the present invention provides a pharmaceutical composition for preventing or treating non-small cell lung cancer (NSCLC), comprising, as an active ingredient, a compound represented by Formula 1 below, a tautomer thereof, a stereoisomer or a mixture thereof, or a pharmaceutically acceptable salt thereof:

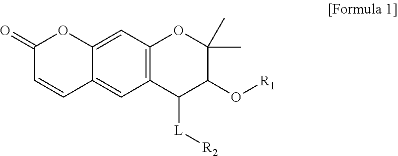

wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

As used herein, the term “pharmaceutically acceptable salt” is as described above.

For example, the compound comprised in the pharmaceutical composition may be: (S,E)-2,2-dimethyl-8-oxo-2,3,4,8-tetrahydropyrano[3,2-g]chromen-3-yl 3-(pyridin-2-yl)acrylate, (S,E)-2,2-dimethyl-8-oxo-2,3,4,8-tetrahydropyrano[3,2-g]chromen-3-yl 2-methyl-3-phenylacrylate, (S,E)-2,2-dimethyl-8-oxo-2,3,4,8-tetrahydropyrano[3,2-g]chromen-3-yl 3-(furan-3-yl)acrylate, (S)-2,2-dimethyl-8-oxo-2,3,4,8-tetrahydropyrano[3,2-g]chromen-3-yl 3-(benzo[d][1,3]dioxol-5-yl)propanoate, (R,E)-2,2-dimethyl-8-oxo-2,3,4,8-tetrahydropyrano[3,2-g]chromen-3-yl 3-(pyridin-2-yl)acrylate, (R,E)-2,2-dimethyl-8-oxo-2,3,4,8-tetrahydropyrano[3,2-g]chromen-3-yl 2-methyl-3-phenylacrylate, (R,E)-2,2-dimethyl-8-oxo-2,3,4,8-tetrahydropyrano[3,2-g]chromen-3-yl 3-(furan-3-yl)acrylate, (R,E)-2,2-dimethyl-8-oxo-2,3,4,8-tetrahydropyrano[3,2-g]chromen-3-yl 3-(4-(trifluoromethyl)phenyl)acrylate, (E)-3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)acrylamide, or (E)-N-((3S,4R)-3-hydroxy-2,2-dimethyl-8-oxo-2,3,4,8-tetrahydropyrano[3,2-g]chromen-4-yl)-3-(4-(trifluoromethyl)phenyl)acrylamide. Specifically, the compound may be (S,E)-2,2-dimethyl-8-oxo-2,3,4,8-tetrahydropyrano[3,2-g]chromen-3-yl 3-(furan-3-yl)acrylate, but is not limited thereto.

As used herein, the term “non-small cell lung cancer (NSCLC)” refers to a type of lung cancer that is distinguished from small cell lung cancer (SCLC) according to histological classification. More specifically, the NSCLC can be further subdivided into adenocarcinoma, squamous cell carcinoma, and large-cell carcinoma of the lung. The non-small cell lung cancer is classified according to the site of occurrence. Squamous cell carcinoma mainly arises in the large bronchi, grows into the bronchial lumen, and clinically causes symptoms of bronchial obstruction. Adenocarcinoma arises in the epithelium of relatively small bronchioles, and large-cell carcinoma mainly occurs near the lung surface, with about half of cases occurring in the large bronchi. Although the sites of occurrence differ, the major symptoms such as coughing, hemoptysis, chest pain, and dyspnea are similar. Therefore, it is difficult to distinguish the type based only on symptoms, and in many cases, patients do not show symptoms in the early stage and visit the hospital only after the disease has progressed. Compared with small cell lung cancer, the non-small cell lung cancer grows relatively slowly and tends to metastasize after spreading to surrounding tissues. Thus, when detected at an early stage, it may be completely cured through surgery. However, in most cases, diagnosis is made at a considerably advanced stage, so only a small number of patients are eligible for curative resection. Accordingly, patients may receive radiation therapy, chemotherapy, or a combination of both.

As used herein, the term “prevention” refers to any activity that inhibits or delays the occurrence, spread, and recurrence of NSCLC through administering the composition of the present invention, and the term “treatment” refers to any activity that improves or beneficially changes a symptom of the disease through administering the composition of the present invention.

Preferably, the pharmaceutical composition according to the present invention may contain, as an active ingredient, a compound represented by Formula 1 or a pharmaceutically acceptable salt thereof in an amount of 0.1 wt % to 75 wt % based on the total weight of the composition, more preferably 1 wt % to 50 wt %.

The composition of the present invention may further comprise a pharmaceutically acceptable carrier, diluent, or excipient, and may be formulated in various forms according to conventional methods suitable for each intended use, such as oral formulations including powders, granules, tablets, capsules, suspensions, emulsions, syrups, and aerosols, or injectable formulations such as sterile injectable solutions. The composition may be administered orally or via various routes including intravenous, intraperitoneal, subcutaneous, rectal, or topical administration. Examples of suitable carriers, excipients, or diluents that may be comprised in such a composition may include lactose, dextrose, sucrose, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methyl hydroxybenzoate, propyl hydroxybenzoate, talc, magnesium stearate, and mineral oil. In addition, the composition of the present invention may further comprise fillers, anti-coagulants, lubricants, moisturizing agents, fragrances, emulsifiers, preservatives, etc.

Solid preparations for oral administration include tablets, pills, powders, granules, capsules, etc., and such solid preparations are formulated by mixing at least one or more excipients, such as starch, calcium carbonate, sucrose, lactose, and gelatin, in the composition. Further, in addition to simple excipients, lubricants such as magnesium stearate and talc may be used.

Examples of oral liquid preparations may include suspensions, solutions, emulsions, syrups, etc., and in addition to commonly used simple diluents such as water and liquid paraffin, various excipients such as wetting agents, sweeteners, flavoring agents, and preservatives may be included.

Preparations for non-oral administration include sterilized aqueous solvents, non-aqueous solvents, suspensions, emulsions, freeze-dried preparations, and suppositories. As non-aqueous solvents and suspending agents, propylene glycol, polyethylene glycol, plant-based oils such as olive oil, injectable esters such as ethyl oleate, etc. may be used. As bases for suppositories, Witepsol, Macrogol, Tween 61, cocoa butter, lauric acid, glycerogelatin, etc. may be used. Meanwhile, the injections may comprise conventional additives such as solubilizing agents, isotonic agents, suspending agents, emulsifiers, stabilizing agents, and preservatives.

In particular, the composition of the present invention is administered in a pharmaceutically effective amount. As used herein, the term “pharmaceutically effective amount” refers to an amount that is sufficient to treat a disease with a reasonable benefit-risk ratio applicable to medical therapy and without causing significant side effects. The effective dose level may be determined based on factors including the patient's health conditions, type and severity of disease, drug activity, sensitivity to the drug, administration method, administration time, route of administration and elimination rate, treatment duration, and combination or concurrent use of drugs, as well as other well-known factors in the medical field. The composition of the present invention may be administered as a stand-alone treatment or in combination with other treatments, and may be administered sequentially or simultaneously with conventional treatments. The composition of the present invention may be administered in single or multiple doses. It is important to administer the minimum amount that can achieve the maximum effect without side effects, taking all the above-mentioned factors into consideration. This may be readily determined by those skilled in the art.

For example, the administered dose may vary depending on factors such as the route of administration, severity of disease, sex, weight, and age, and thus the administered dose does not limit the scope of the present invention by any means.

Specifically, the effective amount of the compound in the composition of the present invention may vary depending on the patient's age, gender, and weight, and may be administered in a range of 1 mg to 100 mg, preferably 5 mg to 60 mg, per kg of body weight, daily or every other day, or divided into 1 to 3 doses per day. However, the administered dose may vary depending on factors such as the route of administration, severity of disease, gender, weight, and age, and thus the administered dose does not limit the scope of the present invention by any means.

The fourth aspect of the present invention provides a composition for combination therapy for preventing or treating NSCLC, comprising, as active ingredients, a compound represented by Formula 1 below, a tautomer thereof, a stereoisomer or a mixture thereof, or a pharmaceutically acceptable salt thereof, and gefitinib:

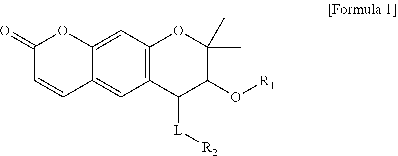

wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

As used herein, the terms “pharmaceutically acceptable salt”, “non-small cell lung cancer (NSCLC)”, “prevention”, and “treatment” are as described above.

For example, the compound comprised in the composition for co-administration may be: (S,E)-2,2-dimethyl-8-oxo-2,3,4,8-tetrahydropyrano[3,2-g]chromen-3-yl 3-(pyridin-2-yl)acrylate, (S,E)-2,2-dimethyl-8-oxo-2,3,4,8-tetrahydropyrano[3,2-g]chromen-3-yl 2-methyl-3-phenylacrylate, (S,E)-2,2-dimethyl-8-oxo-2,3,4,8-tetrahydropyrano[3,2-g]chromen-3-yl 3-(furan-3-yl)acrylate, (S)-2,2-dimethyl-8-oxo-2,3,4,8-tetrahydropyrano[3,2-g]chromen-3-yl 3-(benzo[d][1,3]dioxol-5-yl)propanoate, (R,E)-2,2-dimethyl-8-oxo-2,3,4,8-tetrahydropyrano[3,2-g]chromen-3-yl 3-(pyridin-2-yl)acrylate, (R,E)-2,2-dimethyl-8-oxo-2,3,4,8-tetrahydropyrano[3,2-g]chromen-3-yl 2-methyl-3-phenylacrylate, (R,E)-2,2-dimethyl-8-oxo-2,3,4,8-tetrahydropyrano[3,2-g]chromen-3-yl 3-(furan-3-yl)acrylate, (R,E)-2,2-dimethyl-8-oxo-2,3,4,8-tetrahydropyrano[3,2-g]chromen-3-yl 3-(4-(trifluoromethyl)phenyl)acrylate, (E)-3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)acrylamide, or (E)-N-((3S,4R)-3-hydroxy-2,2-dimethyl-8-oxo-2,3,4,8-tetrahydropyrano[3,2-g]chromen-4-yl)-3-(4-(trifluoromethyl)phenyl)acrylamide. Specifically, the compound may be (S,E)-2,2-dimethyl-8-oxo-2,3,4,8-tetrahydropyrano[3,2-g]chromen-3-yl 3-(furan-3-yl)acrylate, but is not limited thereto.

As used herein, the term “co-administration” means administering two or more drugs in combination. The co-administration may be performed i) to improve the response in patients who do not respond sufficiently to single drug therapy, or ii) to reduce serious or common side effects caused by one component. Therefore, co-administration should provide a potential benefit in improving clinical outcomes of patients. However, based on the nature of administering two or more drugs simultaneously, beneficial drug interactions may occur, such as increased efficacy or reduced adverse drug reactions compared with administration of each drug alone, but the opposite results may also occur. Accordingly, in order to provide safe dosing information, it is necessary to predict possible drug interactions and to develop and comply with co-administration guidelines before co-administration.

In a specific embodiment of the present invention, it was confirmed that the compound of Formula 2, (S,E)-2,2-dimethyl-8-oxo-2,3,4,8-tetrahydropyrano[3,2-g]chromen-3-yl 3-(furan-3-yl)acrylate, exhibited a synergistic cell-killing effect against the non-small cell lung cancer cell line A549 when co-administered with gefitinib, a known therapeutic agent for non-small cell lung cancer.

The fifth aspect of the present invention provides a method for treating non-small cell lung cancer (NSCLC), comprising administering the pharmaceutical composition of the third aspect or the composition for combination therapy of the fourth aspect to a subject in need thereof.

As used herein, the terms “pharmaceutically acceptable salt”, “non-small cell lung cancer (NSCLC)”, and “treatment” are as described above.

As used herein, the term “subject” refers to any animal, including humans in whom non-small cell lung cancer has developed or may develop, and includes monkeys, cattle, horses, sheep, pigs, chickens, turkeys, quails, cats, dogs, mice, rats, rabbits, or guinea pigs. The pharmaceutical composition of the present invention can be administered to the subject to effectively prevent or treat the disease. The pharmaceutical composition of the present invention may be administered in combination with existing therapeutic agents.

The term “administering” as used herein refers to provision of a specified substance to a patient by any appropriate means, and the route of administration of the composition of the present invention may be through any general route that may reach the target tissue. It may be administered intraperitoneally, intravenously, intramuscularly, subcutaneously, intradermally, orally, locally, intranasally, intrapulmonary, and intrarectally, but the route of administration is not limited thereto. In addition, the pharmaceutical composition of the present invention may also be administered by any device capable of transporting the active substance to a target cell. Desirable methods of administration and formulations include intravenous injection, subcutaneous injection, intradermal injection, intramuscular injection, drip injection, etc. The injections may be manufactured using aqueous solvents such as saline solution and Ringer's solution, non-aqueous solvents such as vegetable oils, high-grade fatty acid esters (for example, ethyl oleate), alcohols (for example, ethanol, benzyl alcohol, propylene glycol, or glycerin), etc. The injection may also contain pharmaceutical carriers such as stabilizing agents (for example, ascorbic acid, sodium hydrosulfite, sodium pyrosulfite, BHA, tocopherol, or EDTA), emulsifiers, buffering agents for pH adjustment, and preservatives to inhibit microbial growth (for example, phenylmercuric nitrate, thimerosal, benzalkonium chloride, phenol, cresol, benzyl alcohol, etc.).

In the present invention, the term “therapeutically effective amount”, which is used in conjunction with the active ingredient, refers to the amount of decursin derivative compound or a pharmaceutically acceptable salt thereof that is effective in preventing or treating a target disease.

The pharmaceutical composition of the present invention may further comprise, as an active ingredient, a known drug that is used in preventing or treating each disease in addition to the decursin derivative compound or a pharmaceutically acceptable salt thereof, depending on the type of disease to be prevented or treated. For example, when used in preventing or treating NSCLC, a known drug may be further included in addition to the decursin derivative compound or a pharmaceutically acceptable salt thereof, and the pharmaceutical composition of the present invention may be used in combination with other known treatments for the disease.

### MODE FOR CARRYING OUT THE INVENTION

Hereinafter, the present invention will be described in more detail by way of Examples and Experimental Examples. However, the Examples are provided for illustrative purposes of the present invention, and the scope of the present invention is not limited to the Examples.

### <Experimental Apparatus and Reagents>

The reagents used in the following reactions were purchased from Sigma-Aldrich, Alfa Aesar, and TCI, and were used without further purification. The progress of all reactions was monitored by thin layer chromatography (TLC), for which glass plates coated with silica gel (Merck Silica Gel 60 F254) were used. To visualize the compounds on TLC, a light source emitting ultraviolet (UV) light at 254 nm and 365 nm was used. For more precise monitoring, staining reagents such as ninhydrin, phosphomolybdic acid (PMA), p-anisaldehyde, and iodine, and a heat gun, were used. The obtained compounds were purified by trituration, recrystallization, or column chromatography. Silica gel (Merck, 230-400 mesh) was used for column chromatography. To identify the purified compounds, ¹H NMR and ¹³C NMR spectra were recorded using BrukerAdvance-DXR 400 (400 MHz) and JEOL 500 (500 MHz) spectrometers. In the analysis of the NMR spectra, chemical shifts (6) were reported in ppm relative to tetramethylsilane (TMS) as an internal standard, coupling constants (J) were reported in hertz (Hz), and signal multiplicities were designated as s (singlet), d (doublet), t (triplet), q (quartet), bs s (broad singlet), m (multiplet), and dd (doublet of doublets). High-resolution mass spectra were measured using a Q-Exactive mass spectrometer (ionization mode: ESI+). Enantiomeric excess (% ee) values were determined using HPLC (Agilent 1100) equipped with chiral columns (Chiralpak IK-3, Chiralpak IB-N3).

The roots of Angelica gigas Nakai were extracted three times by boiling with ethanol at 80° C. for 24 hours, followed by filtration and concentration under reduced pressure. The concentrate was extracted with a mixed solution of distilled water and ether, and the ether layer was collected. The obtained ether layer was concentrated under reduced pressure, and a mixed solution of distilled water and tetrahydrofuran (THF), together with an excess amount of NaOH was added, followed by stirring at room temperature for 24 hours. Concentrated hydrochloric acid was then added to adjust to acidity, and the solution was then concentrated. Subsequently, water and dichloromethane (DCM) were added, and DCM was fractionated to obtain an organic layer. The obtained organic layer was dried by adding anhydrous magnesium sulfate and then filtered. Activated carbon was added to the filtrate, which was concentrated under reduced pressure and triturated with DCM. Finally, 31.5 g of decursinol was obtained from 1 kg of the roots of Angelica gigas.

### Preparation Example 2: Preparation of 3-(1H-imidazol-1-yl)propanoic acid

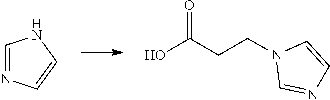 [SMILES: c1c[nH]cn1 ; O=C(O)CCn1ccnc1]

Imidazole (1 g, 14.69 mmol, 1 eq) was dissolved in N,N-dimethylformamide (DMF; 20 mL), followed by the addition of acrylic acid (1.11 mL, 16.16 mmol, 1.1 eq), and the mixture was refluxed for 2 hours and 30 minutes. After completion of the reaction, the mixture was triturated with ethyl acetate (EtOAc), and the filter cake was washed with acetone to afford the title compound as a white solid (1.72 g, 83%).

¹H NMR (400 MHz, DMSO-d₆) δ 12.52 (1H, br s), 7.60 (1H, s), 7.16 (1H, s), 6.86 (1H, s), 4.16 (2H, t, J=6.8 Hz), 2.72 (2H, t, J=6.8 Hz).

### Preparation Example 3: Preparation of (E)-3-(pyridin-2-yl)acrylic acid

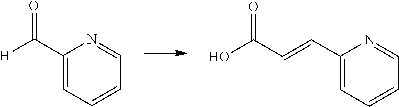 [SMILES: O=Cc1ccccn1 ; O=C(O)/C=C/c1ccccn1]

Malonic acid (0.49 g, 4.67 mmol, 1 eq) and piperidine (0.05 mL, 0.47 mmol, 0.1 eq) were dissolved in pyridine (0.5 mL), followed by slow addition of 2-pyridinecarboxaldehyde (picolinaldehyde; 0.44 mL, 4.67 mmol, 1 eq). The mixture was then refluxed for 2 hours and 30 minutes. After completion of the reaction, the mixture was cooled to room temperature and extracted with EtOAc and distilled water. The extracted organic layer was dried over anhydrous MgSO₄ and concentrated under reduced pressure to remove the solvent. The residue was triturated with DCM to afford the title compound as a light yellow solid (0.14 g, 21%).

¹H NMR (400 MHz, DMSO-d₆) δ 12.64 (1H, br s), 8.64 (4H, d, J=4.0 Hz), 7.88-7.84 (1H, m), 7.33 (1H, d, J=8.0 Hz), 7.60 (1H, d, J=15.6 Hz), 7.41-7.39 (1H, m), 6.83 (1H, d, J=15.6 Hz).

### Preparation Example 4: Preparation of (E)-3-(4-(N,N-dimethylsulfamoyl)phenyl)acrylic acid

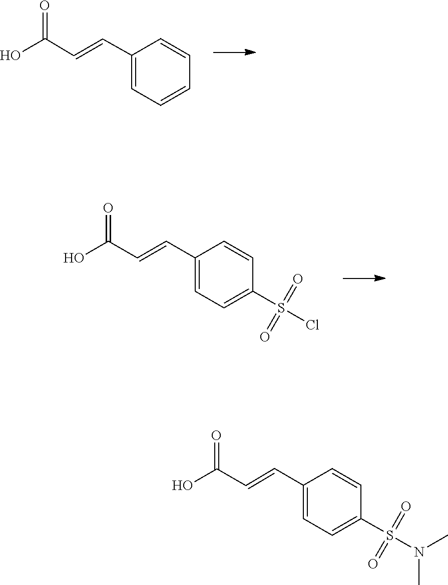 [SMILES: O=C(O)/C=C/c1ccccc1 ; O=C(O)/C=C/c1ccc(S(=O)(=O)Cl)cc1 ; CN(C)S(=O)(=O)c1ccc(/C=C/C(=O)O)cc1]

### Step 1: Preparation of (E)-3-(4-(chlorosulfamoyl)phenyl)acrylic acid

Chlorosulfonic acid (3.6 mL, 54.00 mmol, 8 eq) was cooled to 0° C., and trans-cinnamic acid (1.00 g, 6.75 mmol, 1 eq) was slowly added, followed by reaction for 5 hours. After completion of the reaction, the mixture was quenched with distilled water and triturated, and then filtered. The filter cake was washed with DCM to afford the title compound as a beige solid (1.32 g, 79%).

¹H NMR (400 MHz, DMSO-d₆) δ 13.47 (1H, br s), 7.65 (2H, d, J=8.0 Hz), 7.60 (2H, d, J=8.0 Hz), 7.57 (1H, d, J=16.0 Hz), 6.53 (1H, d, J=16.0 Hz).

### Step 2: Preparation of (E)-3-(4-(N,N-dimethylsulfamoyl)phenyl)acrylic acid

A 40% dimethylamine solution (2.3 mL, 18.24 mmol, 9 eq) was cooled to 0° C., and (E)-3-(4-(chlorosulfamoyl)phenyl)acrylic acid (0.5 g, 2.02 mmol, 1 eq), prepared according to Step 1 of Preparation Example 4, was added, followed by reaction for 17 hours. After completion of the reaction, the reaction mixture was acidified with concentrated hydrochloric acid and filtered. The filter cake was washed with DCM to afford the title compound as a light brown solid (0.39 g, 76%).

¹H NMR (400 MHz, DMSO-d₆) δ 12.65 (1H, br s), 7.96 (2H, d, J=8.0 Hz), 7.75 (2H, d, J=8.0 Hz), 7.67 (1H, d, J=16.0 Hz), 6.71 (1H, d, J=16.0 Hz), 2.62 (6H, s).

### Example 1: Preparation of (S)-2,2-dimethyl-8-oxo-2,3,4,8-tetrahydropyrano[3,2-g]chromen-3-yl 3-(1H-imidazol-1-yl)propanoate

Decursinol (0.20 g, 0.81 mmol, 1 eq), prepared according to Preparation Example 1, and 3-(1H-imidazol-1-yl)propanoic acid (0.14 g, 0.97 mmol, 1.2 eq), prepared according to Preparation Example 2, were dissolved in dry DCM together with DMAP (4-(dimethylamino)pyridine; 0.05 g, 0.14 mmol, 0.5 eq). EDC-HCl (1-ethyl-3-(3-dimethylaminopropyl)carbodiimide hydrochloride, 0.19 g, 0.97 mmol, 1.2 eq) was then added at 0° C., and the mixture was reacted at room temperature for 19 hours. After completion of the reaction, the mixture was extracted with DCM and distilled water. The extracted organic layer was dried over anhydrous MgSO₄, concentrated under reduced pressure to remove the solvent, and purified by flash column chromatography (DCM:MeOH=40:1). The residue was triturated with DCM and ether to afford the title compound as a white solid (0.20 g, 67%).

¹H NMR (400 MHz, CDCl3) δ 8.19 (1H, s), 7.62 (1H, d, J=9.2 Hz), 7.18 (1H, s), 7.13 (1H, s), 7.01 (1H, s), 6.80 (1H, s), 6.25 (1H, d, J=9.2 Hz), 5.09 (1H, t, J=4.4 Hz), 4.40-4.37 (2H, m), 3.17 (1H, dd, J=17.2 Hz and 4.4 Hz), 2.9(1-2).87 (2H, m), 2.78 (1H, dd, J=17.2 Hz and 4.4 Hz), 1.33 (3H, s), 1.32 (3H, s).

### Example 2: Preparation of (S)-2,2-dimethyl-8-oxo-2,3,4,8-tetrahydropyrano[3,2-g]chromen-3-yl 3-(pyridin-2-yl)propanoate

The procedure was performed in a manner similar to that of Example 1, except that 3-(pyridin-2-yl)propanoic acid (0.14 g, 0.89 mmol, 1.1 eq) was used instead of 3-(1H-imidazol-1-yl)propanoic acid, the reaction was carried out for 13 hours, and the product was purified by flash column chromatography (EA:Hex=1:1), to afford the title compound as a colorless viscous liquid (0.18 g, 60%).

¹H NMR (400 MHz, CDCl3) δ 8.47 (1H, d, J=4.4 Hz), 7.59 (1H, d, J=9.6 Hz), 7.54 (1H, t, J=7.6 Hz), 7.12-7.08 (3H, m), 6.78 (1H, s), 6.24 (1H, d, J=9.6 Hz), 5.05 (1H, t, J=4.8 Hz), 3.17-3.07 (3H, m), 2.9(1-2).78 (3H, m), 1.32 (6H, s).

### Example 3: Preparation of (S,E)-2,2-dimethyl-8-oxo-2,3,4,8-tetrahydropyrano[3,2-g]chromen-3-yl 3-(pyridin-2-yl)acrylate

The procedure was performed in a manner similar to that of Example 1, except that (E)-3-(pyridin-2-yl)acrylic acid (0.16 g, 1.06 mmol, 1.3 eq), prepared according to Preparation Example 3, was used instead of 3-(1H-imidazol-1-yl)propanoic acid. The reaction mixture was extracted and the solvent was removed, the product was purified by flash column chromatography (EA:Hex=2:3), and the residue was triturated with ether and n-hexane, to afford the title compound as a white solid (0.12 g, 39%).

¹H NMR (400 MHz, CDCl3) δ 8.63 (1H, s), 7.72-7.66 (2H, m), 7.59 (1H, d, J=8.8 Hz), 7.42 (1H, d, J=8.0 Hz), 7.26 (1H, s), 7.17 (1H, s), 6.92 (1H, d, J=15.6 Hz), 6.83 (1H, s), 6.24 (1H, d, J=10.0 Hz), 5.19 (1H, s), 3.25 (1H, d, J=12.8 Hz), 2.94 (1H, d, J=12.8 Hz), 1.43 (3H, s), 1.40 (3H, s).

### Example 4: Preparation of (S,E)-2,2-dimethyl-8-oxo-2,3,4,8-tetrahydropyrano[3,2-g]chromen-3-yl 2-methyl-3-phenylacrylate

The procedure was performed in a manner similar to that of Example 1, except that (E)-2-methyl-3-phenylacrylic acid (0.07 g, 0.45 mmol, 1.1 eq) was used instead of 3-(1H-imidazol-1-yl)propanoic acid, the reaction was carried out for 23 hours, and the product was purified by flash column chromatography (EA:Hex=1:2), to afford the title compound as a yellow viscous liquid (0.13 g, 85%).

¹H NMR (400 MHz, CDCl3) δ 7.65 (1H, s), 7.61 (1H, J=9.6 Hz), 7.42-7.34 (5H, m), 7.19 (1H, s), 6.84 (1H, s), 6.25 (1H, d, J=9.6 Hz), 5.18 (1H, t, J=4.8 Hz), 3.28 (1H, dd, J=16.8 Hz and 4.4 Hz), 2.98 (1H, dd, J=16.8 Hz and 4.4 Hz), 2.10 (3H, s), 1.46 (3H, s), 1.43 (3H, s).

### Example 5: Preparation of (S,E)-2,2-dimethyl-8-oxo-2,3,4,8-tetrahydropyrano[3,2-g]chromen-3-yl 3-(furan-3-yl)acrylate

The procedure was performed in a manner similar to that of Example 1, except that (E)-3-(furan-3-yl)acrylic acid (0.06 g, 0.45 mmol, 1.1 eq) was used instead of 3-(1H-imidazol-1-yl)propanoic acid. The reaction mixture was extracted and the solvent was removed, the product was purified by flash column chromatography (EA:Hex=1:3), and the residue was triturated with ether and n-hexane, to afford the title compound as a white solid (0.09 g, 59%, 92% ee).

m.p. 128-130° C.;

¹H NMR (400 MHz, CDCl3) δ 7.66 (1H, s), 7.60-7.56 (2H, m), 7.43 (1H, s), 7.19 (1H, s), 6.84 (1H, s), 6.57 (1H, s), 6.25 (1H, d, J=10.0 Hz), 6.15 (1H, d, J=15.6 Hz), 5.19 (1H, s), 3.25 (1H, d, J=16.0 Hz), 2.94 (1H, d, J=16.0 Hz), 1.44 (3H, s), 1.40 (3H, s);

¹³C NMR (400 MHz, CDCl3) 23.4, 24.9, 27.9, 70.0, 76.7, 104.8, 107.3, 112.9, 113.4, 115.7, 117.1, 122.4, 128.8, 135.8, 143.2, 144.6, 144.9, 154.2, 156.4, 161.3, 166.3;
HRMS (ESI+): m/z calcd for C₂₁H₁₈O₆ [M+H]+ 367.1176, found 367.1183.

### Example 6: Preparation of (S)-2,2-dimethyl-8-oxo-2,3,4,8-tetrahydropyrano[3,2-g]chromen-3-yl 3-(benzo[d][1,3]dioxol-5-yl)propanoate

The procedure was performed in a manner similar to that of Example 1, except that 3-(benzo[d][1,3]dioxol-5-yl)propanoic acid (0.09 g, 0.45 mmol, 1.1 eq) was used instead of 3-(1H-imidazol-1-yl)propanoic acid, the reaction was carried out for 5 hours, and the product was purified by flash column chromatography (EA:Hex=1:2) and then triturated with ether, to afford the title compound as a white liquid (0.10 g, 58%).

¹H NMR (400 MHz, CDCl3) δ 7.60 (1H, d, J=9.6 Hz), 7.13 (1H, s), 6.80 (1H, s), 6.68 (1H, d, J=8.0 Hz), 6.63 (1H, s), 6.59 (1H, d, J=8.0 Hz), 6.26 (1H, d, J=9.6 Hz), 5.92 (2H, d, J=2.4 Hz), 5.04 (1H, t, J=4.8 Hz), 3.16 (1H, dd, J=17.6 Hz and 4.4 Hz), 2.85 (2H, t, J=7.6 Hz), 2.78 (1H, dd, J=17.6 Hz and 4.4 Hz), 2.62 (2H, t, J=7.6 Hz), 1.33 (3H, s), 1.32 (3H, s).

### Example 7: Preparation of (S,E)-2,2-dimethyl-8-oxo-2,3,4,8-tetrahydropyrano[3,2-g]chromen-3-yl 3-(benzo[d][1,3]dioxol-5-yl)acrylate

The procedure was performed in a manner similar to that of Example 1, except that 3-(benzo[d][1,3]dioxol-5-yl)acrylic acid (0.10 g, 0.53 mmol, 1.3 eq) was used instead of 3-(1H-imidazol-1-yl)propanoic acid, the reaction was carried out for 18 hours, and the product was purified by flash column chromatography (EA:Hex=2:3) and then triturated with ether and n-hexane, to afford the title compound as a white liquid (0.12 g, 70%).

¹H NMR (400 MHz, CDCl₃) δ 7.62 (1H, s), 7.58 (1H, d, J=6.8 Hz), 7.19 (1H, s), 7.02-6.99 (2H, m), 6.85 (1H, s), 6.81 (1H, d, J=8.0 Hz), 6.27-6.23 (2H, m), 6.02 (2H, s), 5.20 (1H, t, J=4.8 Hz), 3.26 (1H, dd, J=16.8 Hz and 4.4 Hz), 2.95 (1H, dd, J=16.8 Hz and 4.4 Hz), 1.44 (3H, s), 1.40 (3H, s).

### Example 8: Preparation of (S,E)-2,2-dimethyl-8-oxo-2,3,4,8-tetrahydropyrano[3,2-g]chromen-3-yl 3-(4-(N,N-dimethylsulfamoyl)phenyl)acrylate

The procedure was performed in a manner similar to that of Example 1, except that (E)-3-(4-(N,N-dimethylsulfamoyl)phenyl)acrylic acid (0.11 g, 0.45 mmol, 1.1 eq), prepared according to Preparation Example 4, was used instead of 3-(1H-imidazol-1-yl)propanoic acid, the reaction was carried out for 3 hours, and the product was purified by flash column chromatography (EA:Hex=1:1) and then triturated with DCM and n-hexane, to afford the title compound as a white liquid (0.09 g, 45%).

¹H NMR (500 MHz, CDCl₃) δ 7.76 (2H, d, J=6.5 Hz), 7.67 (1H, d, J=16.0 Hz), 7.63 (2H, d, J=8.5 Hz), 7.57 (1H, d, J=9.5 Hz), 7.17 (1H, s), 6.82 (1H, s), 6.50 (1H, d, J=16.0 Hz), 6.23 (1H, d, J=9.5 Hz), 5.20 (1H, t, J=4.5 Hz), 3.25 (1H, dd, J=17.5 Hz and 4.0 Hz), 2.94 (1H, dd, J=17.5 Hz and 4.0 Hz), 2.70 (6H, s), 1.43 (3H, s), 1.38 (3H, s).

### Example 9: Preparation of (S,E)-2,2-dimethyl-8-oxo-2,3,4,8-tetrahydropyrano[3,2-g]chromen-3-yl 3-(4-(trifluoromethyl)phenyl)acrylate

The procedure was performed in a manner similar to that of Example 1, except that (E)-3-(4-(trifluoromethyl)phenyl)acrylic acid (0.10 g, 0.45 mmol, 1.1 eq) was used instead of 3-(1H-imidazol-1-yl)propanoic acid, the reaction was carried out for 4 hours, and the product was purified by flash column chromatography (EA:Hex=1:1) and then triturated with DCM and n-hexane, to afford the title compound as a white liquid (0.10 g, 56%).

¹H NMR (400 MHz, CDCl₃) δ 7.72-7.60 (6H, m), 7.20 (1H, s), 6.86 (1H, s), 6.51 (1H, d, J=16.0 Hz), 6.26 (1H, d, J=9.2 Hz), 5.23 (1H, t, J=4.8 Hz), 3.28 (1H, dd, J=18.0 Hz and 4.8 Hz), 2.97 (1H, dd, J=18.0 Hz and 4.8 Hz), 1.46 (3H, s), 1.41 (3H, s).

### Preparation Example 5: Preparation of 8,8-dimethylpyrano[3,2-g]chromen-2(8H)-one

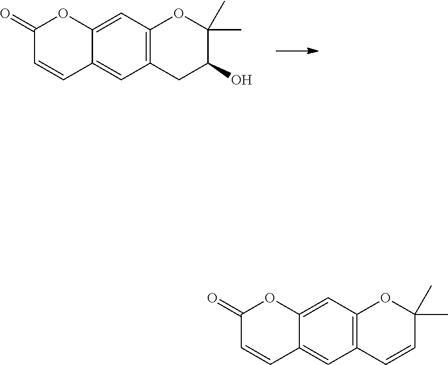 [SMILES: CC1(C)Oc2cc3oc(=O)ccc3cc2C[C@@H]1O ; CC1(C)C=Cc2cc3ccc(=O)oc3cc2O1]

Decursinol (1 g, 4.06 mmol, 1 eq), prepared according to Preparation Example 1, and triphenylphosphine (2.66 g, 10.15 mmol, 2.5 eq) were dissolved in ACN/CCl₄ (14 mL, 1:1, v/v), and the mixture was reacted at 60° C. for 3 hours. After completion of the reaction, the solvent was removed under reduced pressure, and the product was purified by flash column chromatography (EA:Hex=1:3) to afford the title compound as a beige solid (0.78 g, 84%).

¹H NMR (400 MHz, CDCl₃) δ 7.60 (1H, d, J=9.2 Hz), 7.07 (1H, s), 6.74 (1H, s), 6.36 (1H, d, J=10.0 Hz), 6.24 (1H, d, J=9.2 Hz), 5.71 (1H, d, J=10.0 Hz), 1.48 (6H, s).

### Preparation Example 6: Preparation of (1aR,9bR)-2,2-dimethyl-1a,9b-dihydro-2H,6H-oxireno[2,3-c]pyrano[3,2-g]chromen-6-one

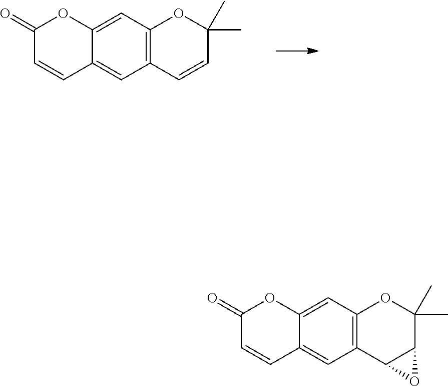 [SMILES: CC1(C)C=Cc2cc3ccc(=O)oc3cc2O1 ; CC1(C)Oc2cc3oc(=O)ccc3cc2[C@H]2O[C@H]21]

33 mL of 15% NaOCl and 13 mL of 0.05 M NaHPO₄ were mixed, and the pH was adjusted to 11.3 by adding 1 M NaOH or 1 M HCl. Separately, 8,8-dimethylpyrano[3,2-g]chromen-2(8H)-one (0.7 g, 3.07 mmol, 1 eq), prepared according to Preparation Example 5, and (R,R)-Jacobsen's catalyst (39 mg, 0.06 mmol, 0.02 eq) were dissolved in DCM (8 mL), and the prepared solution was added thereto, followed by reaction at 0° C. for 72 hours. After completion of the reaction, the mixture was extracted with DCM and distilled water. The extracted organic layer was dried over anhydrous MgSO₄, concentrated under reduced pressure to remove the solvent, and purified by flash column chromatography (EA:Hex=1:3) to afford the title compound as a white solid (0.29 g, 39%, 96% ee).

¹H NMR (400 MHz, CDCl₃) δ 7.65 (1H, d, J=9.6 Hz), 7.48 (1H, s), 6.79 (1H, s), 6.30 (1H, d, J=9.6 Hz), 3.99 (1H, d, J=4.4 Hz), 3.56 (1H, d, J=4.4 Hz), 1.63 (3H, s), 1.33 (3H, s).

### Preparation Example 7: Preparation of (R)-7-hydroxy-8,8-dimethyl-7,8-dihydropyrano[3,2-g]chromen-2(6H)-one

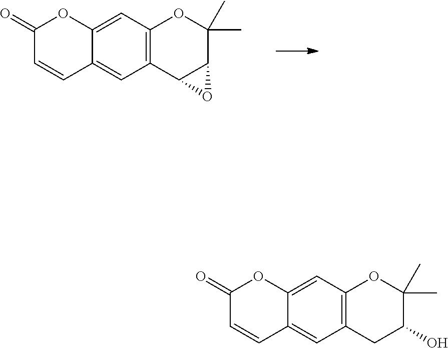 [SMILES: CC1(C)Oc2cc3oc(=O)ccc3cc2[C@H]2O[C@H]21 ; CC1(C)Oc2cc3oc(=O)ccc3cc2C[C@H]1O]

(1aR,9bR)-2,2-dimethyl-1a,9b-dihydro-2H,6H-oxireno[2,3-c]pyrano[3,2-g]chromen-6-one (0.2 g, 0.82 mmol, 1 eq), prepared according to Preparation Example 6, and NaCNBH₄ (0.2 g, 0.82 mmol, 1 eq) were dissolved in THF (15 mL) and cooled to 0° C. BF₄OEt₂ (0.10 mL, 0.82 mmol, 1 eq) was then added to the solution, and the mixture was further reacted for 10 minutes. After completion of the reaction, the mixture was extracted with EtOAc and distilled water. The extracted organic layer was dried over anhydrous MgSO₄, concentrated under reduced pressure to remove the solvent, and purified by flash column chromatography (EA) to afford the title compound as a white solid (0.20 g, 90%).

¹H NMR (400 MHz, CDCl₃) δ 7.60 (1H, d, J=9.6 Hz), 7.20 (1H, s), 6.80 (1H, s), 6.24 (1H, d, J=9.6 Hz), 3.89 (1H, q, J=5.6 Hz), 3.14 (1H, dd, J=16.8 Hz and 4.8 Hz), 2.86 (1H, dd, J=16.8 Hz and 4.8 Hz), 1.85 (1H, d, J=6.8 Hz), 1.41 (3H, s), 1.38 (3H, s).

### Example 10: Preparation of (R)-2,2-dimethyl-8-oxo-2,3,4,8-tetrahydropyrano[3,2-g]chromen-3-yl 3-(1H-imidazol-1-yl)propanoate

The mixture was extracted, dried, and purified in a manner similar to that of Example 1, except that (R)-7-hydroxy-8,8-dimethyl-7,8-dihydropyrano[3,2-g]chromen-2(6H)-one (0.1 g, 0.41 mmol, 1 eq), prepared according to Preparation Example 7, was used instead of decursinol, and the reaction was carried out for 20 hours, to afford the title compound as a white solid (0.09 g, 62%).

¹H NMR (400 MHz, CDCl₃) δ 7.61 (1H, d, J=9.6 Hz), 7.47 (1H, s), 7.16 (1H, s), 7.03 (1H, s), 6.89 (1H, s), 6.80 (1H, s), 6.26 (1H, d, J=9.6 Hz), 5.09 (1H, t, J=4.4 Hz), 4.26 (2H, t, J=6.4 Hz), 3.16 (1H, dd, J=17.6 Hz and 4.4 Hz), 2.83-2.73 (3H, m), 1.32 (3H, s), 1.31 (3H, s).

### Example 11: Preparation of (R)-2,2-dimethyl-8-oxo-2,3,4,8-tetrahydropyrano[3,2-g]chromen-3-yl 3-(pyridin-2-yl)propanoate

The procedure was performed in a manner similar to that of Example 10, except that 3-(pyridin-2-yl)propanoic acid (0.04 g, 0.27 mmol, 1.1 eq) was used instead of 3-(1H-imidazol-1-yl)propanoic acid, the reaction was carried out for 21 hours, and the product was purified by flash column chromatography (EA:Hex=1:1), to afford the title compound as a colorless viscous liquid (0.02 g, 18%).

¹H NMR (400 MHz, CDCl₃) δ 8.48 (1H, d, J=4.8 Hz), 7.59 (1H, d, J=9.6 Hz), 7.55 (1H, t, J=7.6 Hz), 7.12-7.08 (3H, m), 6.79 (1H, s), 6.25 (1H, d, J=9.6 Hz), 5.06 (1H, t, J=4.8 Hz), 3.18-3.08 (3H, m), 2.9(1-2).78 (3H, m), 1.33 (6H, s).

### Example 12: Preparation of (R,E)-2,2-dimethyl-8-oxo-2,3,4,8-tetrahydropyrano[3,2-g]chromen-3-yl 3-(pyridin-2-yl)acrylate

The procedure was performed in a manner similar to that of Example 10, except that (E)-3-(pyridin-2-yl)acrylic acid (0.06 g, 0.42 mmol, 1.1 eq) was used instead of 3-(1H-imidazol-1-yl)propanoic acid. The reaction was carried out for 13 hours, and the reaction mixture was extracted and the solvent was removed. The product was then purified by flash column chromatography (EA:Hex=2:3), and the residue was triturated with ether and n-hexane, to afford the title compound as a white solid (0.03 g, 27%).

¹H NMR (400 MHz, CDCl₃) δ 8.65 (1H, s), 7.74-7.68 (2H, m), 7.61 (1H, d, J=9.6 Hz), 7.44 (1H, d, J=6.8 Hz), 7.28 (1H, s), 7.19 (1H, s), 6.94 (1H, d, J=15.6 Hz), 6.84 (1H, s), 6.26 (1H, d, J=9.2 Hz), 5.21 (1H, t, J=4.8 Hz), 3.27 (1H, dd, J=17.2 Hz and 4.8 Hz), 2.96 (1H, dd, J=17.2 Hz and 4.8 Hz), 1.44 (3H, s), 1.41 (3H, s).

### Example 13: Preparation of (S,E)-2,2-dimethyl-8-oxo-2,3,4,8-tetrahydropyrano[3,2-g]chromen-3-yl 2-methyl-3-phenylacrylate

The procedure was performed in a manner similar to that of Example 10, except that (E)-2-methyl-3-phenylacrylic acid (0.04 g, 0.27 mmol, 1.1 eq) was used instead of 3-(1H-imidazol-1-yl)propanoic acid, the reaction was carried out for 12 hours, and the product was purified by flash column chromatography (EA:Hex=1:1), to afford the title compound as a yellow viscous liquid (0.05 g, 53%).

¹H NMR (400 MHz, CDCl₃) δ 7.65 (1H, s), 7.61 (1H, J=9.6 Hz), 7.42-7.34 (5H, m), 7.19 (1H, s), 6.84 (1H, s), 6.25 (1H, d, J=9.6 Hz), 5.18 (1H, t, J=4.8 Hz), 3.28 (1H, dd, J=16.8 Hz and 4.4 Hz), 2.98 (1H, dd, J=16.8 Hz and 4.4 Hz), 2.10 (3H, s), 1.46 (3H, s), 1.43 (3H, s).

### Example 14: Preparation of (R,E)-2,2-dimethyl-8-oxo-2,3,4,8-tetrahydropyrano[3,2-g]chromen-3-yl 3-(furan-3-yl)acrylate

The procedure was performed in a manner similar to that of Example 10, except that (E)-3-(furan-3-yl)acrylic acid (0.04 g, 0.31 mmol, 1.1 eq) was used instead of 3-(1H-imidazol-1-yl)propanoic acid and that the reaction was carried out for 13 hours.

The reaction mixture was extracted and the solvent was removed, the product was purified by flash column chromatography (EA:Hex=1:3), and the residue was triturated with ether and n-hexane, to afford the title compound as a white solid (0.03 g, 33%, 96% ee).

¹H NMR (400 MHz, CDCl₃) δ 7.66 (1H, s), 7.62-7.56 (2H, m), 7.43 (1H, s), 7.19 (1H, s), 6.84 (1H, s), 6.57 (1H, s), 6.25 (1H, d, J=9.6 Hz), 6.15 (1H, d, J=16.0 Hz), 5.19 (1H, t, J=4.4 Hz), 3.25 (1H, dd, J=16.8 Hz and 4.4 Hz), 2.94 (1H, dd, J=16.8 Hz and 4.4 Hz), 1.44 (3H, s), 1.40 (3H, s).

### Example 15: Preparation of (R)-2,2-dimethyl-8-oxo-2,3,4,8-tetrahydropyrano[3,2-g]chromen-3-yl 3-(benzo[d][1,3]dioxol-5-yl)propanoate

The procedure was performed in a manner similar to that of Example 10, except that 3-(benzo[d][1,3]dioxol-5-yl)propanoic acid (0.05 g, 0.27 mmol, 1.1 eq) was used instead of 3-(1H-imidazol-1-yl)propanoic acid, the reaction was carried out for 5 hours, and the product was purified by flash column chromatography (EA:Hex=1:3) and then triturated with ether and n-hexane, to afford the title compound as a white liquid (0.06 g, 59%).

¹H NMR (400 MHz, CDCl₃) δ 7.60 (1H, d, J=9.6 Hz), 7.13 (1H, s), 6.80 (1H, s), 6.68 (1H, d, J=7.6 Hz), 6.62 (1H, s), 6.59 (1H, d, J=7.6 Hz), 6.26 (1H, d, J=9.6 Hz), 5.92-5.91 (2H, m), 5.04 (1H, t, J=4.8 Hz), 3.16 (1H, dd, J=16.8 Hz and 4.4 Hz), 2.85 (2H, t, J=7.6 Hz), 2.78 (1H, dd, J=16.8 Hz and 4.4 Hz), 2.62 (2H, t, J=7.6 Hz), 1.33 (3H, s), 1.32 (3H, s).

### Example 16: Preparation of (R,E)-2,2-dimethyl-8-oxo-2,3,4,8-tetrahydropyrano[3,2-g]chromen-3-yl 3-(benzo[d][1,3]dioxol-5-yl)acrylate

The procedure was performed in a manner similar to that of Example 10, except that 3-(benzo[d][1,3]dioxol-5-yl)acrylic acid (0.08 g, 0.42 mmol, 1.1 eq) was used instead of 3-(1H-imidazol-1-yl)propanoic acid, the reaction was carried out for 16 hours, and the product was purified by flash column chromatography (EA:Hex=2:3) and then triturated with ether and n-hexane, to afford the title compound as a white liquid (0.10 g, 62%).

¹H NMR (400 MHz, CDCl₃) δ 7.62 (1H, s), 7.58 (1H, d, J=6.8 Hz), 7.19 (1H, s), 7.02-6.99 (2H, m), 6.85 (1H, s), 6.81 (1H, d, J=7.6 Hz), 6.27-6.23 (2H, m), 6.02 (2H, s), 5.20 (1H, t, J=4.8 Hz), 3.26 (1H, dd, J=17.2 Hz and 4.4 Hz), 2.95 (1H, dd, J=17.2 Hz and 4.4 Hz), 1.44 (3H, s), 1.40 (3H, s).

### Example 17: Preparation of (R,E)-2,2-dimethyl-8-oxo-2,3,4,8-tetrahydropyrano[3,2-g]chromen-3-yl 3-(4-(N,N-dimethylsulfamoyl)phenyl)acrylate

The procedure was performed in a manner similar to that of Example 1, except that (E)-3-(4-(N,N-dimethylsulfamoyl)phenyl)acrylic acid (0.07 g, 0.27 mmol, 1.1 eq) was used instead of 3-(1H-imidazol-1-yl)propanoic acid, the reaction was carried out for 12 hours, and the product was purified by flash column chromatography (EA:Hex=1:1) and then triturated with DCM and n-hexane, to afford the title compound as a white liquid (0.03 g, 25%).

¹H NMR (400 MHz, CDCl₃) δ 7.79 (2H, d, J=8.0 Hz), 7.73 (1H, s), 7.68-7.65 (2H, m), 7.60 (1H, d, J=9.6 Hz), 7.19 (1H, s), 6.85 (1H, s), 6.53 (1H, d, J=16.0 Hz), 6.26 (1H, d, J=9.6 Hz), 5.23 (1H, t, J=4.4 Hz), 3.28 (1H, dd, J=17.6 Hz and 4.4 Hz), 2.97 (1H, dd, J=17.6 Hz and 4.4 Hz), 2.74 (6H, s), 1.46 (3H, s), 1.42 (3H, s).

### Example 18: Preparation of (R,E)-2,2-dimethyl-8-oxo-2,3,4,8-tetrahydropyrano[3,2-g]chromen-3-yl 3-(4-(trifluoromethyl)phenyl)acrylate

The procedure was performed in a manner similar to that of Example 10, except that (E)-3-(4-(trifluoromethyl)phenyl)acrylic acid (0.06 g, 0.27 mmol, 1.1 eq) was used instead of 3-(1H-imidazol-1-yl)propanoic acid, the reaction was carried out for 12 hours, and the product was purified by flash column chromatography (EA:Hex=1:1) and then triturated with n-hexane, to afford the title compound as a white liquid (0.02 g, 20%).

¹H NMR (400 MHz, CDCl₃) δ 7.72-7.60 (6H, m), 7.20 (1H, s), 6.86 (1H, s), 6.51 (1H, d, J=16.0 Hz), 6.26 (1H, d, J=9.6 Hz), 5.23 (1H, t, J=4.4 Hz), 3.28 (1H, dd, J=17.2 Hz and 4.4 Hz), 2.97 (1H, dd, J=17.2 Hz and 4.4 Hz), 1.46 (3H, s), 1.41 (3H, s).

### Preparation Example 8: Preparation of (1aS,9bS)-2,2-dimethyl-1a,9b-dihydro-2H,6H-oxireno[2,3-c]pyrano[3,2-g]chromen-6-one

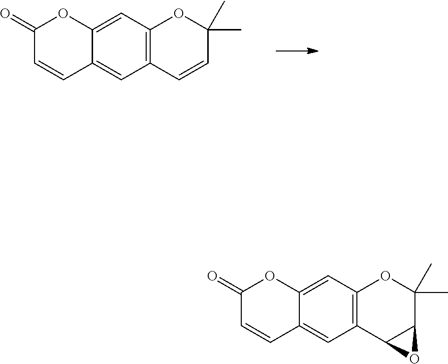 [SMILES: CC1(C)C=Cc2cc3ccc(=O)oc3cc2O1 ; CC1(C)Oc2cc3oc(=O)ccc3cc2[C@@H]2O[C@@H]21]

The procedure was performed in a manner similar to that of Preparation Example 6, except that (S,S)-Jacobsen's catalyst (0.20 g, 0.31 mmol, 0.02 eq) was used instead of (R,R)-Jacobsen's catalyst, to afford the title compound as a white solid (1.88 g, 50%, 95% ee).

¹H NMR (400 MHz, CDCl₃) δ 7.65 (1H, d, J=9.6 Hz), 7.48 (1H, s), 6.79 (1H, s), 6.30 (1H, d, J=9.6 Hz), 3.99 (1H, d, J=4.4 Hz), 3.56 (1H, d, J=4.4 Hz), 1.63 (3H, s), 1.32 (3H, s).

### Preparation Example 9: Preparation of (6R,7S)-6-amino-7-hydroxy-8,8-dimethyl-7,8-dihydropyrano[3,2-g]chromen-2(6H)-one

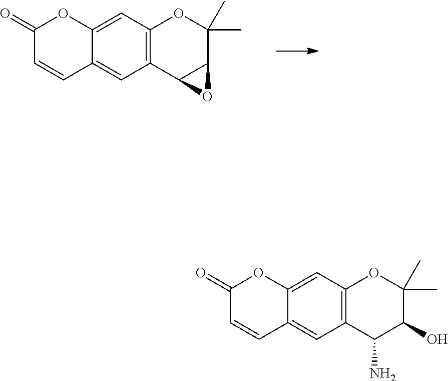 [SMILES: CC1(C)Oc2cc3oc(=O)ccc3cc2[C@@H]2O[C@@H]21 ; CC1(C)Oc2cc3oc(=O)ccc3cc2[C@@H](N)[C@@H]1O]

(1 aS,9bS)-2,2-dimethyl-1a,9b-dihydro-2H,6H-oxireno[2,3-c]pyrano[3,2-g]chromen-6-one (0.1 g, 0.41 mmol, 1 eq), prepared according to Preparation Example 8, was dissolved in methanol (3 mL), and 7 M ammonia solution (0.58 mL, 4.09 mmol, 10 eq) was added thereto. The mixture was reacted for 13 hours. After completion of the reaction, the solvent was removed under reduced pressure, and the product was purified by flash column chromatography (DCM:MeOH=9:1) to afford the title compound as a light yellow solid (0.03 g, 31%).

¹H NMR (400 MHz, CDCl₃) δ 7.64 (1H, d, J=9.6 Hz), 7.56 (1H, s), 6.75 (1H, s), 6.26 (1H, d, J=9.6 Hz), 3.72 (1H, d, J=9.6 Hz), 3.39 (1H, d, J=9.6 Hz), 1.79 (3H, br s), 1.55 (3H, s), 1.27 (3H, s).

### Example 19: Preparation of N-((3S,4R)-3-hydroxy-2,2-dimethyl-8-oxo-2,3,4,8-tetrahydropyrano[3,2-g]chromen-4-yl)-3-(1H-imidazol-1-yl)propanamide

(6R,7S)-6-amino-7-hydroxy-8,8-dimethyl-7,8-dihydropyrano[3,2-g]chromen-2(6H)-one (0.06 g, 0.23 mmol, 1 eq), prepared according to Preparation Example 9, was dissolved in DMF (2 mL) together with 3-(1H-imidazol-1-yl)propanoic acid and 1-[bis(dimethylamino)methylene]-1H-1,2,3-triazolo[4,5-b]pyridinium 3-oxide hexafluorophosphate (hexafluorophosphate azabenzotriazole tetramethyl uronium, HATU; 0.10 g, 0.25 mmol, 1.1 eq). N,N-diisopropylethylamine (DIPEA; 0.1 mL, 0.57 mmol, 2.5 eq) was then added, and the mixture was reacted for 15 hours. After completion of the reaction, the mixture was extracted with EtOAc and distilled water. The extracted organic layer was dried over anhydrous MgSO₄, concentrated under reduced pressure to remove the solvent, and triturated with acetone to afford the title compound as a white solid (0.04 g, 43%).

¹H NMR (400 MHz, DMSO-d₆) δ 8.36 (1H, d, J=8.8 Hz), 7.99 (1H, d, J=9.6 Hz), 7.66 (1H, s), 7.21 (1H, s), 6.94 (1H, s), 6.84 (1H, s), 6.75 (1H, s), 6.30 (1H, d, J=9.6 Hz), 5.67 (1H, d, J=5.6 Hz), 4.83 (1H, t, J=9.2 Hz), 4.41-4.34 (1H, m), 4.26-4.20 (1H, m), 3.49-3.45 (1H, m), 2.7(1-2).64 (2H, m), 1.39 (3H, s), 1.16 (3H, s).

### Example 20: Preparation of N-((3S,4R)-3-hydroxy-2,2-dimethyl-8-oxo-2,3,4,8-tetrahydropyrano[3,2-g]chromen-4-yl)-3-(pyridin-2-yl)propanamide

The procedure was performed in a manner similar to that of Example 19, except that 3-(pyridin-2-yl)propanoic acid (0.04 g, 0.25 mmol, 1.1 eq) was used instead of 3-(1H-imidazol-1-yl)propanoic acid, the reaction was carried out for 4 hours, and the product was purified by flash column chromatography (DCM:MeOH=20:1) and then triturated with DCM and n-hexane, to afford the title compound as a white liquid (0.04 g, 46%).

¹H NMR (400 MHz, DMSO-d₆) δ 8.49 (1H, d, J=3.6 Hz), 8.30 (1H, d, J=7.6 Hz), 7.90 (1H, d, J=9.6 Hz), 7.75-7.71 (1H, m), 7.32 (1H, d, J=7.6 Hz), 7.28 (1H, s), 7.25-7.22 (1H, m), 6.76 (1H, s), 6.27 (1H, d, J=9.6 Hz), 5.66 (1H, d, J=5.6 Hz), 4.86 (1H, t, J=9.2 Hz), 3.60-3.52 (1H, m), 3.16-3.03 (2H, m), 2.72-2.58 (2H, m), 1.41 (3H, s), 1.18 (3H, s).

### Example 21: Preparation of (E)-N-((3S,4R)-3-hydroxy-2,2-dimethyl-8-oxo-2,3,4,8-tetrahydropyrano[3,2-g]chromen-4-yl)-3-(pyridin-2-yl)acrylamide

The procedure was performed in a manner similar to that of Example 19, except that (E)-3-(pyridin-2-yl)acrylic acid (0.04 g, 0.25 mmol, 1.1 eq) was used instead of 3-(1H-imidazol-1-yl)propanoic acid, the reaction was carried out for 11 hours, and the product was purified by flash column chromatography (DCM:MeOH=20:1) and then triturated with THF and n-hexane, to afford the title compound as a white liquid (0.04 g, 45%).

¹H NMR (400 MHz, DMSO-d₆) δ 8.73 (1H, d, J=8.4 Hz), 8.63 (1H, d, J=4.0 Hz), 8.04 (1H, d, J=9.6 Hz), 7.88-7.84 (1H, m), 7.63 (1H, d, J=8.4 Hz), 7.57 (1H, d, J=15.6 Hz), 7.45 (1H, s), 7.39-7.36 (1H, m), 7.16 (1H, d, J=15.6 Hz), 6.80 (1H, s), 6.24 (1H, d, J=9.6 Hz), 5.76 (1H, d, J=5.6 Hz), 5.00 (1H, t, J=9.2 Hz), 3.63-3.59 (1H, m), 1.44 (3H, s), 1.22 (3H, s).

### Example 22: Preparation of (E)-N-((3S,4R)-3-hydroxy-2,2-dimethyl-8-oxo-2,3,4,8-tetrahydropyrano[3,2-g]chromen-4-yl)-2-methyl-3-phenylacrylamide

The procedure was performed in a manner similar to that of Example 19, except that (E)-2-methyl-3-phenylacrylic acid (0.04 g, 0.25 mmol, 1.1 eq) was used instead of 3-(1H-imidazol-1-yl)propanoic acid, the reaction was carried out for 2 hours, and the product was purified by flash column chromatography (DCM:MeOH=20:1) and then triturated with ether and n-hexane, to afford the title compound as a white liquid (0.04 g, 48%).

¹H NMR (400 MHz, CDCl₃) δ 7.61 (1H, d, J=9.6 Hz), 7.55 (1H, s), 7.45-7.38 (5H, m), 7.36 (1H, s), 6.79 (1H, s), 6.36 (1H, d, J=8.0 Hz), 6.25 (1H, d, J=9.6 Hz), 5.27 (1H, t, J=8.8 Hz), 4.28 (1H, d, J=3.2 Hz), 3.81 (1H, dd, J=8.8 Hz and 3.2 Hz), 2.20 (3H, s), 1.57 (3H, s), 1.35 (3H, s).

### Example 23: Preparation of (E)-3-(furan-3-yl)-N-((3S,4R)-3-hydroxy-2,2-dimethyl-8-oxo-2,3,4,8-tetrahydropyrano[3,2-g]chromen-4-yl)acrylamide

The procedure was performed in a manner similar to that of Example 19, except that (E)-3-(furan-3-yl)acrylic acid (0.04 g, 0.25 mmol, 1.1 eq) was used instead of 3-(1H-imidazol-1-yl)propanoic acid, the reaction was carried out for 3 hours, and the product was purified by flash column chromatography (DCM:MeOH=20:1) and then triturated with ether and n-hexane, to afford the title compound as a white liquid (0.05 g, 62%).

¹H NMR (400 MHz, DMSO-d₆) δ 8.48 (1H, d, J=8.8 Hz), 8.07-8.03 (2H, m), 7.75 (1H, s), 7.48-7.43 (2H, m), 6.79-6.68 (2H, m), 6.41 (1H, d, J=15.6 Hz), 6.26-6.22 (1H, m), 5.73-5.69 (1H, m), 4.98 (1H, t, J=9.2 Hz), 3.65-3.57 (1H, m), 1.43 (3H, s), 1.21 (3H, s).

### Example 24: Preparation of 3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)propanamide

The procedure was performed in a manner similar to that of Example 10, except that 3-(benzo[d][1,3]dioxol-5-yl)propanoic acid (0.03 g, 0.15 mmol, 1.1 eq) was used instead of 3-(1H-imidazol-1-yl)propanoic acid, the reaction was carried out for 2 hours and 30 minutes, and the product was purified by flash column chromatography (DCM:MeOH=60:1) and then triturated with ether and n-hexane, to afford the title compound as a white liquid (0.04 g, 73%).

¹H NMR (400 MHz, DMSO-d₆) δ 8.24 (1H, d, J=8.8 Hz), 7.83 (1H, d, J=9.6 Hz), 7.15 (1H, s), 6.88-6.84 (2H, m), 6.76-6.72 (2H, m), 6.29 (1H, d, J=9.6 Hz), 5.97 (2H, d, J=9.6 Hz), 5.63 (1H, d, J=5.6 Hz), 4.84 (1H, t, J=9.2 Hz), 3.58-3.49 (1H, m), 2.92-2.79 (2H, m), 2.56-2.47 (2H, m), 1.40 (3H, s), 1.18 (3H, s).

### Example 25: Preparation of (E)-3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)acrylamide

The procedure was performed in a manner similar to that of Example 19, except that (E)-3-(benzo[d][1,3]dioxol-5-yl)acrylic acid (0.05 g, 0.25 mmol, 1.1 eq) was used instead of 3-(1H-imidazol-1-yl)propanoic acid, the reaction was carried out for 12 hours, and the product was purified by flash column chromatography (DCM:MeOH=20:1) and then triturated with ether and n-hexane, to afford the title compound as a white liquid (0.06 g, 57%).

¹H NMR (400 MHz, DMSO-d₆) δ 8.47 (1H, d, J=8.8 Hz), 8.04 (1H, d, J=9.6 Hz), 7.49-7.44 (2H, m), 7.18-7.10 (2H, m), 6.98 (1H, d, J=8.0 Hz), 6.79 (1H, s), 6.54 (1H, d, J=16.0 Hz), 6.26-6.22 (1H, m), 6.08 (2H, s), 5.71 (1H, d, J=5.6 Hz), 4.99 (1H, t, J=9.2 Hz), 3.65-3.58 (1H, m), 1.43 (3H, s), 1.22 (3H, s).

### Example 26: Preparation of (E)-3-(4-(N,N-dimethylsulfamoyl)phenyl)-N-((3S,4R)-3-hydroxy-2,2-dimethyl-8-oxo-2,3,4,8-tetrahydropyrano[3,2-g]chromen-4-yl)acrylamide

The procedure was performed in a manner similar to that of Example 19, except that (E)-3-(4-(N,N-dimethylsulfamoyl)phenyl)acrylic acid (0.06 g, 0.25 mmol, 1.1 eq) was used instead of 3-(1H-imidazol-1-yl)propanoic acid, the reaction was carried out for 3 hours, and the product was purified by flash column chromatography (DCM:MeOH=60:1) and then triturated with ether and n-hexane, to afford the title compound as a white liquid (0.04 g, 34%).

¹H NMR (400 MHz, DMSO-d₆) δ 8.68 (1H, d, J=8.8 Hz), 8.03 (1H, d, J=10.0 Hz), 7.87-7.79 (4H, m), 7.64 (1H, d, J=16.0 Hz), 7.46 (1H, s), 6.87-6.77 (2H, m), 6.28 (1H, d, J=9.2 Hz), 5.78-5.72 (1H, m), 5.01 (1H, t, J=9.2 Hz), 3.68-3.61 (1H, m), 2.64 (6H, s), 1.44 (3H, s), 1.23 (3H, s).

### Example 27: Preparation of (E)-N-((3S,4R)-3-hydroxy-2,2-dimethyl-8-oxo-2,3,4,8-tetrahydropyrano[3,2-g]chromen-4-yl)-3-(4-(trifluoromethyl)phenyl)acrylamide

The procedure was performed in a manner similar to that of Example 19, except that (E)-3-(4-(trifluoromethyl)phenyl)acrylic acid (0.03 g, 0.15 mmol, 1.1 eq) was used instead of 3-(1H-imidazol-1-yl)propanoic acid, the reaction was carried out for 2 hours and 30 minutes, and the product was purified by flash column chromatography (DCM:MeOH=60:1) and then triturated with ether and n-hexane, to afford the title compound as a white liquid (0.04 g, 67%).

¹H NMR (400 MHz, DMSO-d₆) δ 8.69 (1H, d, J=9.2 Hz), 8.03 (1H, d, J=9.2 Hz), 7.84-7.79 (4H, m), 7.62 (1H, d, J=16.0 Hz), 7.46 (1H, s), 6.83 (1H, d, J=16.0 Hz), 6.78 (1H, d, J=12.8 Hz), 6.25 (1H, d, J=9.6 Hz), 5.76 (1H, d, J=5.6 Hz), 5.00 (1H, t, J=8.8 Hz), 3.63-3.59 (1H, m), 1.44 (3H, s), 1.22 (3H, s).

### Example 28: Preparation of (S)-2,2-dimethyl-8-oxo-2,3,4,8-tetrahydropyrano[3,2-g]chromen-3-yl 3-(furan-3-yl)propanoate

(2E)-3-(furan-3-yl)-2-propenoic acid (0.30 g, 2.17 mmol, 1 eq) and 10% palladium catalyst (0.02 g, 0.02 mmol, 0.01 eq) were dissolved in methanol, and hydrogen gas was introduced. The mixture was stirred for 30 minutes. After completion of the reaction, the palladium catalyst was filtered off through a celite pad to afford a pure 1:1 mixture of 3-(furan-3-yl)propanoic acid and 3-(tetrahydrofuran-3-yl)propanoic acid (>99%).

The procedure was performed in a manner similar to that of Example 1, except that 3-(furan-3-yl)propanoic acid (0.10 g, 0.71 mmol, 1.1 eq) was used instead of 3-(1H-imidazol-1-yl)propanoic acid, the reaction was carried out for 7 hours, and the product was purified by flash column chromatography (EA:Hex=1:1), to afford the title compound as a colorless viscous liquid (0.08 g, 64%).

¹H NMR (500 MHz, CDCl₃) δ 7.57 (d, J=9.5 Hz, 1H), 7.29 (s, 1H), 7.15 (s, 1H), 7.11 (s, 1H), 6.78 (s, 1H), 6.22 (d, J=9.5 Hz, 1H), 6.21 (s, 1H), 5.03 (t, J=4.8 Hz, 1H), 3.14 (dd, J=17.1, 4.8 Hz, 1H), 2.77 (dd, J=17.1, 4.8 Hz, 1H), 2.72 (t, J=7.4 Hz, 2H).

### Example 29: Preparation of (S)-2,2-dimethyl-8-oxo-2,3,4,8-tetrahydropyrano[3,2-g]chromen-3-yl 4-(dimethylamino)phenylacetate

The procedure was performed in a manner similar to that of Example 1, except that 4-(dimethylaminophenyl)acetic acid (0.15 g, 0.84 mmol, 1.2 eq) was used instead of 3-(1H-imidazol-1-yl)propanoic acid, the reaction was carried out for 10 hours, and the product was purified by flash column chromatography (EA:Hex=1:1), to afford the title compound as a colorless viscous liquid (0.09 g, 32%).

¹H NMR (500 MHz, CDCl₃) δ 7.56 (d, J=9.5 Hz, 1H), 7.08 (s, 1H), 7.04 (d, J=8.7 Hz, 2H), 6.78 (s, 1H), 6.59 (d, J=8.7 Hz, 2H), 6.22 (d, J=9.5 Hz, 1H), 4.99 (t, J=5.0 Hz, 1H), 3.48 (s, 2H), 3.12 (dd, J=17.2, 5.0 Hz, 1H), 2.89 (s, 6H), 2.78 (dd, J=17.2, 5.0 Hz, 1H), 1.31 (s, 3H), 1.29 (s, 3H).

### Example 30: Preparation of (S,E)-2,2-dimethyl-8-oxo-2,3,4,8-tetrahydropyrano[3,2-g]chromen-3-yl 3-(4-(dimethylamino)phenyl)acrylate

Malonic acid (0.49 g, 4.7 mmol, 1 eq) and triethylamine (0.85 mL, 6.1 mmol, 1.5 eq) were stirred in toluene for 3 minutes, followed by addition of 4-dimethylaminobenzaldehyde (0.70 g, 4.7 mmol, 1 eq) and piperidine (0.08 mL, 0.8 mmol, 0.17 eq). The mixture was stirred at 80° C. for 24 hours. After completion of the reaction, toluene was removed using a rotary evaporator, and the mixture was extracted with EtOAc and an aqueous sodium bicarbonate solution. The extracted aqueous layer was adjusted to pH 3 by adding hydrochloric acid solution at 0° C., and the precipitate formed was filtered to afford (E)-3-(4-(dimethylamino)phenyl)acrylic acid as a light yellow solid (0.45 g, 50%).

The procedure was performed in a manner similar to that of Example 1, except that (E)-3-(4-(dimethylamino)phenyl)acrylic acid (0.2 g, 1.04 mmol, 1.5 eq) was used instead of 3-(1H-imidazol-1-yl)propanoic acid, the reaction was carried out for 8 hours, and the product was purified by flash column chromatography (EA:Hex=1:1), to afford the title compound as a colorless viscous liquid (0.05 g, 19%).

¹H NMR (500 MHz, CDCl₃) δ 7.59 (d, J=15.8 Hz, 1H), 7.57 (d, J=9.4 Hz, 1H), 7.37 (d, J=8.9 Hz, 2H), 7.16 (s, 1H), 6.82 (s, 1H), 6.62 (d, J=8.9 Hz, 2H), 6.22 (d, J=9.4 Hz, 1H), 6.17 (d, J=15.8 Hz, 1H), 5.16 (t, J=4.8 Hz, 1H), 3.21 (dd, J=17.2, 4.8 Hz, 1H), 3.00 (s, 6H), 2.92 (d, J=17.2, 4.8 Hz, 1H), 1.42 (s, 3H), 1.37 (s, 3H).

### Experimental Example 1: Cell Cultivation

A549 (human lung cancer cells) were obtained from the American Type Culture Collection (ATCC) and used. The cells were cultured in a medium containing 10% FBS, 1% penicillin (10,000 units/mL), and streptomycin (10 mg/mL), or in Dulbecco's Modified Eagle's Medium (DMEM), at 37° C. under 5% CO₂ conditions.

### Experimental Example 2: Cytotoxicity Evaluation

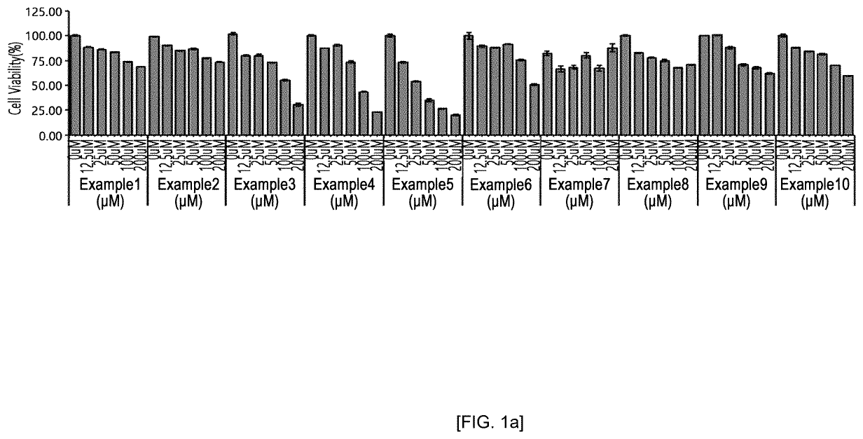

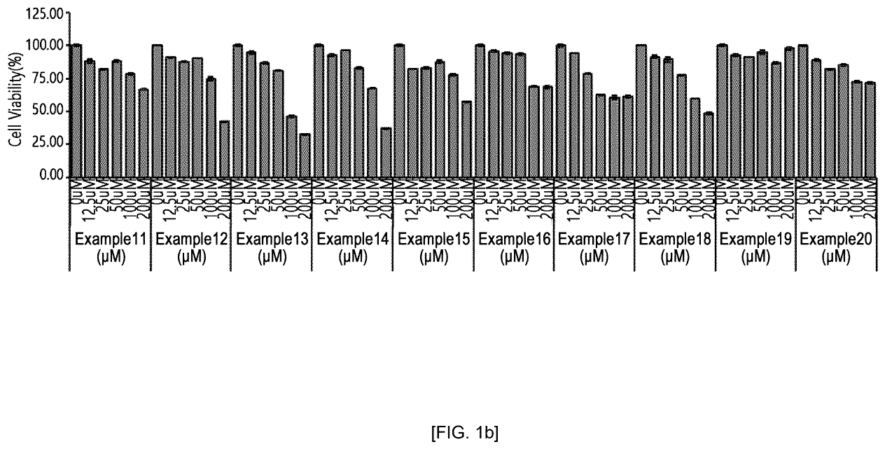

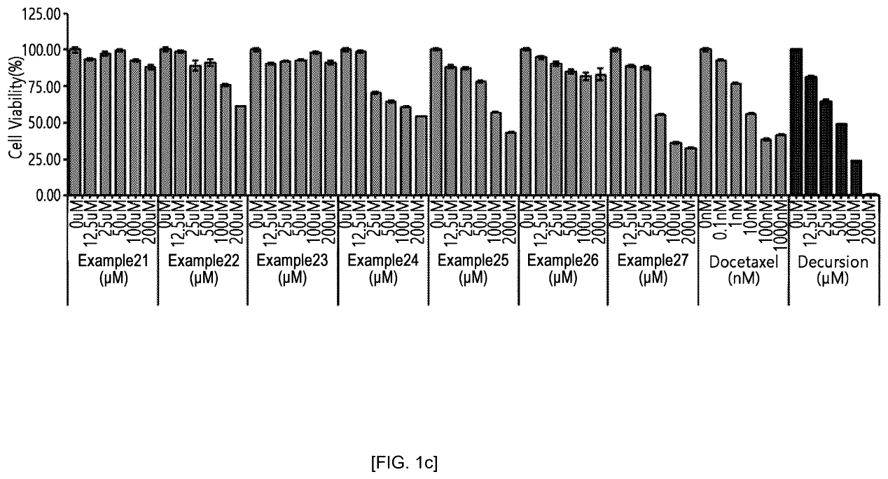

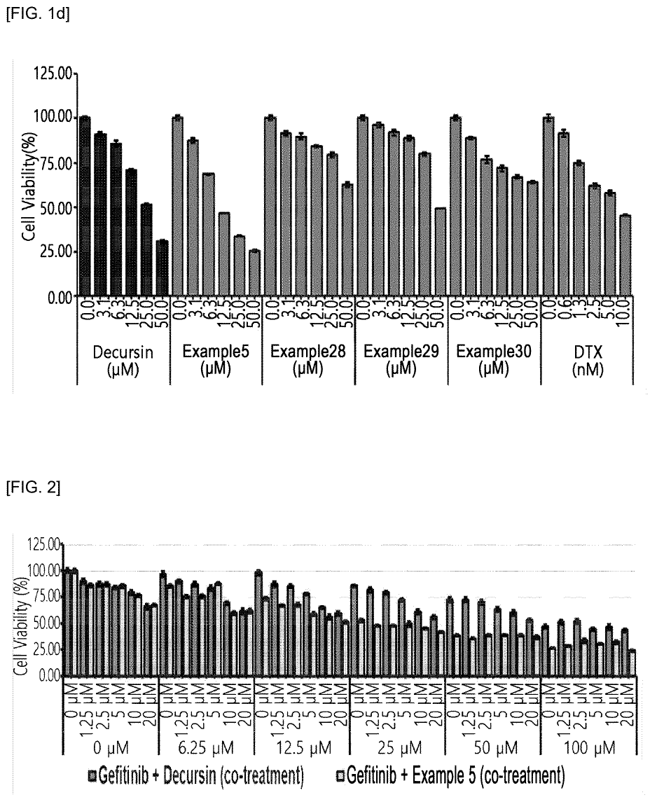

To measure cytotoxicity, an MTT assay was used. Specifically, A549 cells cultured according to Experimental Example 1 were seeded into a 96-well plate at a density of 2.5×10⁴ cells/mL in 100 μL, and incubated for 24 hours. Thereafter, the cells were treated with either the compounds prepared according to Examples 1 to 27 or, as comparative agents, decursin or docetaxel. The prepared compounds and decursin were each administered to the A549 cell line at concentrations of 0 μM (untreated), 12.5 μM, 25 μM, 50 μM, 100 μM, and 200 μM, while docetaxel was administered at concentrations of 0 nM (untreated), 0.1 nM, 1 nM, 10 nM, 100 nM, and 1,000 nM, followed by incubation for an additional 48 hours. Then, 20 μL of MTT solution was added (Method 1). Alternatively, the compounds prepared according to Examples 28 to 30, the compound of Example 5 as a reference compound or, as comparative agents, decursin or docetaxel, were used. The prepared compounds, the compound of Example 5, and decursin were each administered at concentrations of 0 μM (untreated), 3.125 μM, 6.25 μM, 12.5 μM, 25 μM, and 50 μM, while docetaxel was administered at concentrations of 0 nM (untreated), 0.6 nM, 1.25 nM, 2.5 nM, 5 nM, and 10 nM, and treated in the same manner as described in Method 1 (Method 2). The cells treated with MTT solution were incubated for 2 hours, the medium was removed, and the formazan blue crystals formed by the cells were treated with an MTT lysis buffer, followed by incubation in a cell incubator for 24 hours. The MTT lysis buffer was prepared by mixing N,N-dimethylformamide with water in a 1:1 ratio to give a solution containing 20% SIDS (sodium dodecyl sulfate) at the final concentration. The optical density at 590 nm was measured using a microplate reader (FIGS. 1a to 1d). Based on the measured values, the cytotoxicity (IC₅₀) against A549 was calculated, and the results are disclosed in Table 1 together with the structural formulae.

TABLE 1

| | | Cytotoxicity |
|---|---|---|
| | | Against A549 |
| Compound | Structural Formula | (IC₅₀, μM) |
| Example 1 | 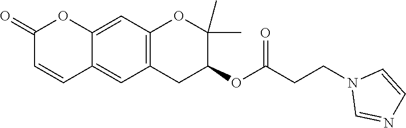 [SMILES: CC1(C)Oc2cc3oc(=O)ccc3cc2C[C@@H]1OC(=O)CCn1ccnc1] | >200 |
| Example 2 | 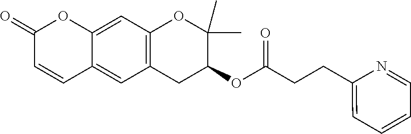 [SMILES: CC1(C)Oc2cc3oc(=O)ccc3cc2C[C@@H]1OC(=O)CCc1ccccn1] | >200 |
| Example 3 | 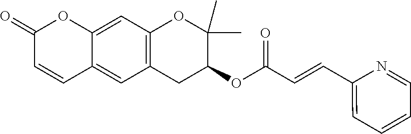 [SMILES: CC1(C)Oc2cc3oc(=O)ccc3cc2C[C@@H]1OC(=O)/C=C/c1ccccn1] | 113.14 |
| Example 4 | 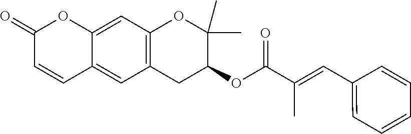 [SMILES: C/C(=C\c1ccccc1)C(=O)O[C@H]1Cc2cc3ccc(=O)oc3cc2OC1(C)C] | 80.66 |
| Example 5 | 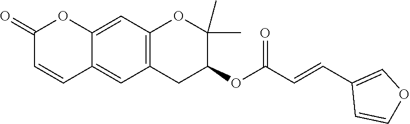 [SMILES: CC1(C)Oc2cc3oc(=O)ccc3cc2C[C@@H]1OC(=O)/C=C/c1ccoc1] | 30.28/7.88* |
| Example 6 | 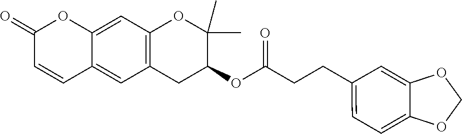 [SMILES: CC1(C)Oc2cc3oc(=O)ccc3cc2C[C@@H]1OC(=O)CCc1ccc2c(c1)OCO2] | 218.82 |
| Example 7 | 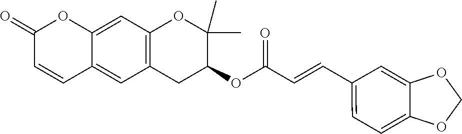 [SMILES: CC1(C)Oc2cc3oc(=O)ccc3cc2C[C@@H]1OC(=O)/C=C/c1ccc2c(c1)OCO2] | >200 |
| Example 8 | 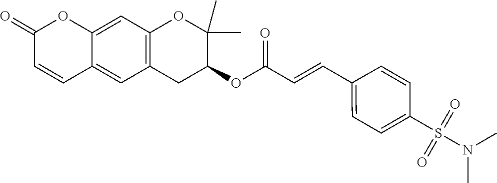 [SMILES: CN(C)S(=O)(=O)c1ccc(/C=C/C(=O)O[C@H]2Cc3cc4ccc(=O)oc4cc3OC2(C)C)cc1] | >200 |
| Example 9 | 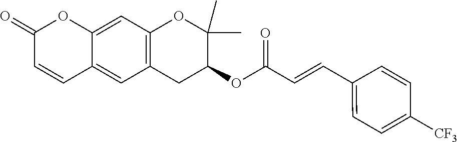 [SMILES: CC1(C)Oc2cc3oc(=O)ccc3cc2C[C@@H]1OC(=O)/C=C/c1ccc(C(F)(F)F)cc1] | >200 |
| Example 10 | 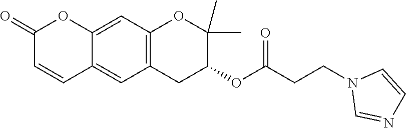 [SMILES: CC1(C)Oc2cc3oc(=O)ccc3cc2C[C@H]1OC(=O)CCn1ccnc1] | >200 |
| Example 11 | 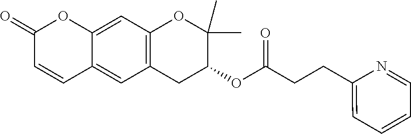 [SMILES: CC1(C)Oc2cc3oc(=O)ccc3cc2C[C@H]1OC(=O)CCc1ccccn1] | >200 |
| Example 12 | 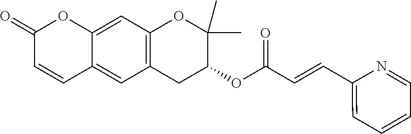 [SMILES: CC1(C)Oc2cc3oc(=O)ccc3cc2C[C@H]1OC(=O)/C=C/c1ccccn1] | 183.84 |
| Example 13 | 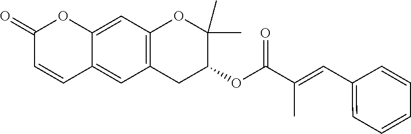 [SMILES: C/C(=C\c1ccccc1)C(=O)O[C@@H]1Cc2cc3ccc(=O)oc3cc2OC1(C)C] | 107.58 |
| Example 14 | 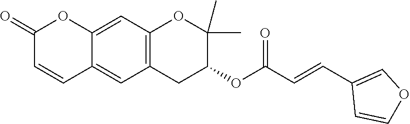 [SMILES: CC1(C)Oc2cc3oc(=O)ccc3cc2C[C@H]1OC(=O)/C=C/c1ccoc1] | 151.59 |
| Example 15 | 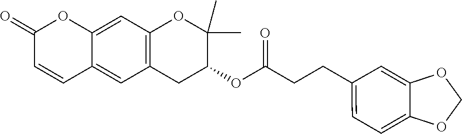 [SMILES: CC1(C)Oc2cc3oc(=O)ccc3cc2C[C@H]1OC(=O)CCc1ccc2c(c1)OCO2] | >200 |
| Example 16 | 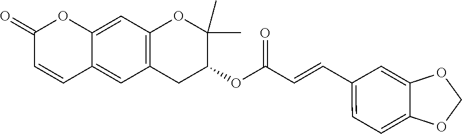 [SMILES: CC1(C)Oc2cc3oc(=O)ccc3cc2C[C@H]1OC(=O)/C=C/c1ccc2c(c1)OCO2] | >200 |
| Example 17 | 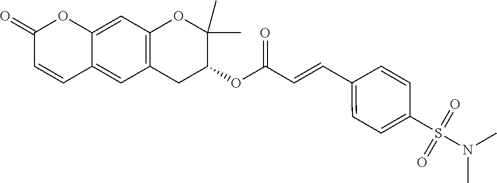 [SMILES: CN(C)S(=O)(=O)c1ccc(/C=C/C(=O)O[C@@H]2Cc3cc4ccc(=O)oc4cc3OC2(C)C)cc1] | >200 |
| Example 18 | 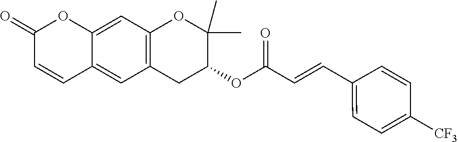 [SMILES: CC1(C)Oc2cc3oc(=O)ccc3cc2C[C@H]1OC(=O)/C=C/c1ccc(C(F)(F)F)cc1] | 171.79 |
| Example 19 | 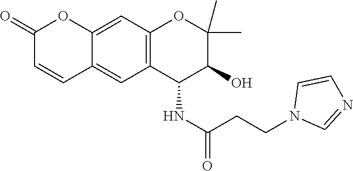 [SMILES: CC1(C)Oc2cc3oc(=O)ccc3cc2[C@@H](NC(=O)CCn2ccnc2)[C@@H]1O] | >200 |
| Example 20 | 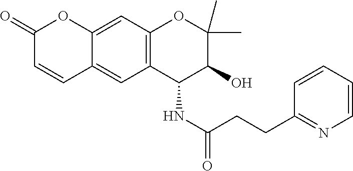 [SMILES: CC1(C)Oc2cc3oc(=O)ccc3cc2[C@@H](NC(=O)CCc2ccccn2)[C@@H]1O] | >200 |
| Example 21 | 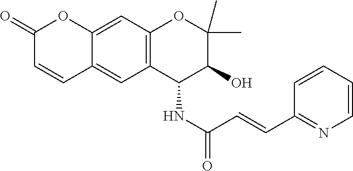 [SMILES: CC1(C)Oc2cc3oc(=O)ccc3cc2[C@@H](NC(=O)/C=C/c2ccccn2)[C@@H]1O] | >200 |
| Example 22 | 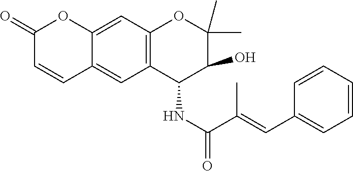 [SMILES: C/C(=C\c1ccccc1)C(=O)N[C@@H]1c2cc3ccc(=O)oc3cc2OC(C)(C)[C@H]1O] | >200 |
| Example 23 | 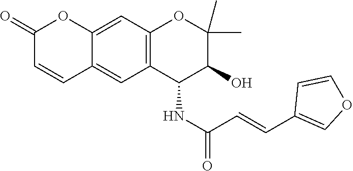 [SMILES: CC1(C)Oc2cc3oc(=O)ccc3cc2[C@@H](NC(=O)/C=C/c2ccoc2)[C@@H]1O] | >200 |
| Example 24 | 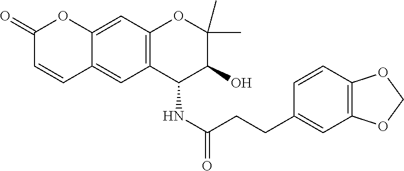 [SMILES: CC1(C)Oc2cc3oc(=O)ccc3cc2[C@@H](NC(=O)CCc2ccc3c(c2)OCO3)[C@@H]1O] | >200 |
| Example 25 | 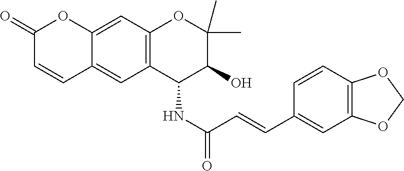 [SMILES: CC1(C)Oc2cc3oc(=O)ccc3cc2[C@@H](NC(=O)/C=C/c2ccc3c(c2)OCO3)[C@@H]1O] | 151.21 |
| Example 26 | 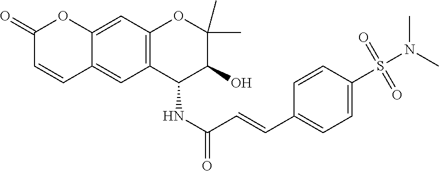 [SMILES: CN(C)S(=O)(=O)c1ccc(/C=C/C(=O)N[C@@H]2c3cc4ccc(=O)oc4cc3OC(C)(C)[C@H]2O)cc1] | >200 |
| Example 27 | 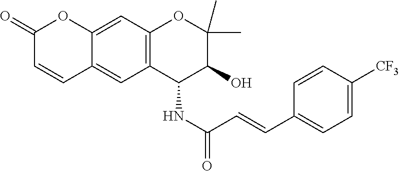 [SMILES: CC1(C)Oc2cc3oc(=O)ccc3cc2[C@@H](NC(=O)/C=C/c2ccc(C(F)(F)F)cc2)[C@@H]1O] | 73.35 |
| Example 28 | 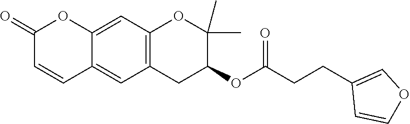 [SMILES: CC1(C)Oc2cc3oc(=O)ccc3cc2C[C@@H]1OC(=O)CCc1ccoc1] | 96.11* |
| Example 29 | 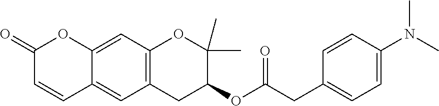 [SMILES: CN(C)c1ccc(CC(=O)O[C@H]2Cc3cc4ccc(=O)oc4cc3OC2(C)C)cc1] | 52.40* |
| Example 30 | 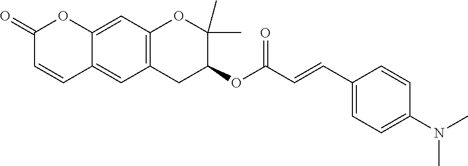 [SMILES: CN(C)c1ccc(/C=C/C(=O)O[C@H]2Cc3cc4ccc(=O)oc4cc3OC2(C)C)cc1] | 150.75* |
| Decursin | 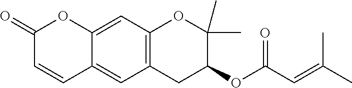 [SMILES: CC(C)=CC(=O)O[C@H]1Cc2cc3ccc(=O)oc3cc2OC1(C)C] | 43.55/30.35* |

*Results measured by Method 2; absence of a mark indicates results measured by Method 1.

As shown in Table 1, decursin as the control and the decursin derivatives prepared according to Examples 1 to 30 exhibited, albeit to varying degrees, a concentration-dependent increase in cytotoxicity across all compounds, as shown in FIG. 1. Although all of these compounds share the common core structure of 2,2-dimethyl-8-oxo-2,3,4,8-tetrahydropyrano[3,2-g]chromen, they exhibited different cytotoxicities against A549 cells. In particular, it was confirmed that the compound of Example 5 exhibited a superior cell-killing effect against the human lung cancer cell line A549 compared to decursin.

### Experimental Example 3: Study on Structure and Activity Relationship (SAR)

The relationship between the structures of the synthesized decursin derivatives and their biological activities was investigated to identify trends. As a result, it was confirmed that, among the newly synthesized 30 decursin derivatives, those derivatives in which the chirality at the chiral center at the 3-position of the 2,2-dimethyl-8-oxo-2,3,4,8-tetrahydropyrano[3,2-g]chromen core was maintained without inversion, consistent with that of decursin, exhibited superior cell-killing effects against the human lung cancer cell line A549.

### Experimental Example 4: Effect of Co-Administration with Gefitinib

The cytotoxicity test against A549 cells conducted in Experimental Example 1 demonstrated that the compound of Example 5 exhibited the most potent effect, and even superior activity compared to decursin as a control. Nevertheless, the compound of Example 5, when used as a single agent, was somewhat limited in its efficacy, and therefore its potential as a co-administration agent with existing non-small cell lung cancer therapeutic agents was examined. To this end, gefitinib (Iressa) was selected, and the compound of Example 5, together with decursin for comparison, was co-administered to A549 cells, and the cytotoxicity thereof was measured. To further evaluate dose dependency, gefitinib was administered at concentrations of 0 μM (untreated), 1.25 μM, 2.5 μM, 5 μM, 10 μM, and 20 μM, in combination with either decursin or the decursin derivative compounds of Examples 1 to 30, each administered at concentrations of 0 μM (untreated), 6.25 μM, 12.5 μM, 25 μM, 50 μM, and 100 μM. The relative cell viability was measured by setting the viability of the untreated group, i.e., cells not treated with either gefitinib or decursin/decursin derivative, to 100%, and the results are shown in FIG. 2. Cell viability was determined using an MTT assay. As shown in FIG. 2, when the decursin derivative compound of Example 5 was co-administered with gefitinib, cytotoxicity at the same concentration combinations was superior to that observed with co-administration of decursin and gefitinib. In particular, as the concentration of the decursin derivative increased to 12.5 μM or higher, the difference in cell-killing effect became more pronounced.

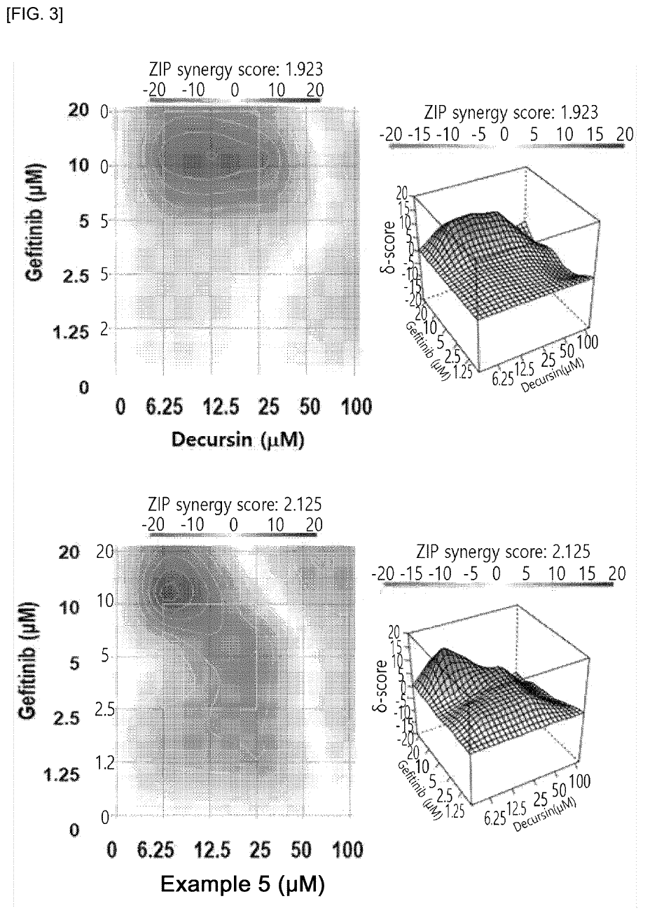

Furthermore, the synergy score of the cytotoxicity of the combination of gefitinib with decursin or with the decursin derivative compound of Example 5 against A549 cells was calculated using the Synergyfinder webtool, and the results are shown in FIG. 3. A calculated synergy score of 10 or more was considered to indicate a synergistic effect, a score between −10 and 10 was considered to indicate an additive effect, and a score below −10 was considered to indicate an antagonistic effect. Based on the analysis of the synergy scores at different concentrations in FIG. 3, the combination of 10 μM gefitinib and 6.25 μM of the decursin derivative compound of Example 5 exhibited the highest synergy score, approaching 15, indicating that this combination exerts a remarkable synergistic effect.

From the above description, those skilled in the art to which the present invention pertains would understand that the present invention can be implemented in other specific forms without changing its technical concept or essential features. In this regard, the foregoing examples should be understood as illustrative and not limited in any way. The scope of the present invention should be interpreted to include all modifications or modified forms derived from the meaning and scope of the claims as set forth below, as well as equivalent concepts, rather than from the detailed description above.

1. A compound represented by Formula 1 below, a tautomer thereof, a stereoisomer or a mixture thereof, or a pharmaceutically acceptable salt thereof: 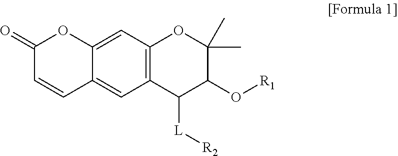
wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

2. The compound, tautomer thereof, stereoisomer thereof or mixture thereof, or pharmaceutically acceptable salt thereof according to claim 1,
wherein when L is a single bond, R₂ is hydrogen, and when L is —NH—, R₁ is hydrogen.

3. The compound, tautomer thereof, stereoisomer thereof or mixture thereof, or pharmaceutically acceptable salt thereof according to claim 1,
wherein any one of R₁ and R₂ is hydrogen and the other is C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl.

4. The compound, tautomer thereof, stereoisomer thereof or mixture thereof, or pharmaceutically acceptable salt thereof according to claim 1,
wherein any one of R₁ and R₂ is hydrogen and the other is 1-methyl-2-phenylethenylcarbonyl, 2-phenylethenylcarbonyl, trifluoromethyl-substituted 2-phenylethenylcarbonyl, dimethylaminosulfonyl-substituted 2-phenylethenylcarbonyl, dimethylamino-substituted 2-phenylethenylcarbonyl, phenylmethylcarbonyl, dimethylamino-substituted phenylmethylcarbonyl, (pyridinylethyl)carbonyl, (benzo[d][1,3]dioxolylethyl)carbonyl, (imidazolylethyl)carbonyl, (furanylethyl)carbonyl, (pyridinylethenyl)carbonyl, (benzo[d][1,3]dioxolylethenyl)carbonyl, (imidazolylethenyl)carbonyl, or (furanylethenyl)carbonyl.

5. The compound, tautomer thereof, stereoisomer thereof or mixture thereof, or pharmaceutically acceptable salt thereof according to claim 1, wherein the compound is
1. (S)-2,2-dimethyl-8-oxo-2,3,4,8-tetrahydropyrano[3,2-g]chromen-3-yl 3-(1H-imidazol-1-yl)propanoate,
2. (S)-2,2-dimethyl-8-oxo-2,3,4,8-tetrahydropyrano[3,2-g]chromen-3-yl 3-(pyridin-2-yl)propanoate,
3. (S,E)-2,2-dimethyl-8-oxo-2,3,4,8-tetrahydropyrano[3,2-g]chromen-3-yl 3-(pyridin-2-yl)acrylate,
4. (S,E)-2,2-dimethyl-8-oxo-2,3,4,8-tetrahydropyrano[3,2-g]chromen-3-yl 2-methyl-3-phenylacrylate,
5. (S,E)-2,2-dimethyl-8-oxo-2,3,4,8-tetrahydropyrano[3,2-g]chromen-3-yl 3-(furan-3-yl)acrylate,
6. (S)-2,2-dimethyl-8-oxo-2,3,4,8-tetrahydropyrano[3,2-g]chromen-3-yl 3-(benzo[d][1,3]dioxol-5-yl)propanoate,
7. (S,E)-2,2-dimethyl-8-oxo-2,3,4,8-tetrahydropyrano[3,2-g]chromen-3-yl 3-(benzo[d][1,3]dioxol-5-yl)acrylate,
8. (S,E)-2,2-dimethyl-8-oxo-2,3,4,8-tetrahydropyrano[3,2-g]chromen-3-yl 3-(4-(N,N-dimethylsulfamoyl)phenyl)acrylate,
9. (S,E)-2,2-dimethyl-8-oxo-2,3,4,8-tetrahydropyrano[3,2-g]chromen-3-yl 3-(4-(trifluoromethyl)phenyl)acrylate,
10. (R)-2,2-dimethyl-8-oxo-2,3,4,8-tetrahydropyrano[3,2-g]chromen-3-yl 3-(1H-imidazol-1-yl)propanoate,
11. (R)-2,2-dimethyl-8-oxo-2,3,4,8-tetrahydropyrano[3,2-g]chromen-3-yl 3-(pyridin-2-yl)propanoate,
12. (R,E)-2,2-dimethyl-8-oxo-2,3,4,8-tetrahydropyrano[3,2-g]chromen-3-yl 3-(pyridin-2-yl)acrylate,
13. (R,E)-2,2-dimethyl-8-oxo-2,3,4,8-tetrahydropyrano[3,2-g]chromen-3-yl 2-methyl-3-phenylacrylate,
14. (R,E)-2,2-dimethyl-8-oxo-2,3,4,8-tetrahydropyrano[3,2-g]chromen-3-yl 3-(furan-3-yl)acrylate,
15. (R)-2,2-dimethyl-8-oxo-2,3,4,8-tetrahydropyrano[3,2-g]chromen-3-yl 3-(benzo[d][1,3]dioxol-5-yl)propanoate,
16. (R,E)-2,2-dimethyl-8-oxo-2,3,4,8-tetrahydropyrano[3,2-g]chromen-3-yl 3-(benzo[d][1,3]dioxol-5-yl)acrylate,
17. (R,E)-2,2-dimethyl-8-oxo-2,3,4,8-tetrahydropyrano[3,2-g]chromen-3-yl 3-(4-(N,N-dimethylsulfamoyl)phenyl)acrylate,
18. (R,E)-2,2-dimethyl-8-oxo-2,3,4,8-tetrahydropyrano[3,2-g]chromen-3-yl 3-(4-(trifluoromethyl)phenyl)acrylate,
19. N-((3S,4R)-3-hydroxy-2,2-dimethyl-8-oxo-2,3,4,8-tetrahydropyrano[3,2-g]chromen-4-yl)-3-(1H-imidazol-1-yl)propanamide,
20. N-((3S,4R)-3-hydroxy-2,2-dimethyl-8-oxo-2,3,4,8-tetrahydropyrano[3,2-g]chromen-4-yl)-3-(pyridin-2-yl)propanamide,
21. (E)-N-((3S,4R)-3-hydroxy-2,2-dimethyl-8-oxo-2,3,4,8-tetrahydropyrano[3,2-g]chromen-4-yl)-3-(pyridin-2-yl)acrylamide,
22. (E)-N-((3S,4R)-3-hydroxy-2,2-dimethyl-8-oxo-2,3,4,8-tetrahydropyrano[3,2-g]chromen-4-yl)-2-methyl-3-phenylacrylamide,
23. (E)-3-(furan-3-yl)-N-((3S,4R)-3-hydroxy-2,2-dimethyl-8-oxo-2,3,4,8-tetrahydropyrano[3,2-g]chromen-4-yl)acrylamide,
24. 3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)propanamide,
25. (E)-3-(benzo[d][1,3]dioxol-5-yl)-N-((3S,4R)-3-hydroxy-2,2-dimethyl-8-oxo-2,3,4,8-tetrahydropyrano[3,2-g]chromen-4-yl)acrylamide,
26. (E)-3-(4-(N,N-dimethylsulfamoyl)phenyl)-N-((3S,4R)-3-hydroxy-2,2-dimethyl-8-oxo-2,3,4,8-tetrahydropyrano[3,2-g]chromen-4-yl)acrylamide,
27. (E)-N-((3S,4R)-3-hydroxy-2,2-dimethyl-8-oxo-2,3,4,8-tetrahydropyrano[3,2-g]chromen-4-yl)-3-(4-(trifluoromethyl)phenyl)acrylamide,
28. (S)-2,2-dimethyl-8-oxo-2,3,4,8-tetrahydropyrano[3,2-g]chromen-3-yl 3-(furan-3-yl)propanoate,
29. (S)-2,2-dimethyl-8-oxo-2,3,4,8-tetrahydropyrano[3,2-g]chromen-3-yl 4-(dimethylamino)phenylacetate, or
30. (S,E)-2,2-dimethyl-8-oxo-2,3,4,8-tetrahydropyrano[3,2-g]chromen-3-yl 3-(4-(dimethylamino)phenyl)acrylate.

6. A method for preparing a compound represented by Formula 1 below, a tautomer thereof, a stereoisomer thereof or a mixture thereof, or a pharmaceutically acceptable salt thereof, comprising:
a step (1-1) of reacting (S)-7-hydroxy-8,8-dimethyl-7,8-dihydropyrano[3,2-g]chromen-2(6H)-one (decursinol), or (R)-7-hydroxy-8,8-dimethyl-7,8-dihydropyrano[3,2-g]chromen-2(6H)-one with R1-OH in the presence of 4-(dimethylamino)pyridine (DMAP) and 1-ethyl-3-(3-dimethylaminopropyl)carbodiimide hydrochloride (EDC-HCl), or
a step (1-2) of reacting (6R,7S)-6-amino-7-hydroxy-8,8-dimethyl-7,8-dihydropyrano[3,2-g]chromen-2(6H)-one with R₂—OH in the presence of 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium; HATU) and N,N-diisopropylethylamine (DIPEA): 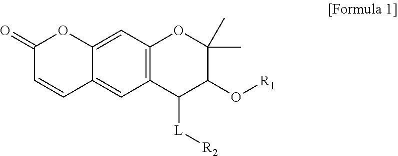
wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

7. The method according to claim 6,
wherein the step (1-1) is performed for 30 minutes to 48 hours by heating to 10° C. to 40° C. following dissolving the reactants in an organic solvent at −5° C. to 10° C.

8. The method according to claim 6,
wherein the step (1-2) is performed in an organic solvent for 30 minutes to 48 hours.

9. The method according to claim 6,
wherein the (S)-7-hydroxy-8,8-dimethyl-7,8-dihydropyrano[3,2-g]chromen-2(6H)-one is separated from an Angelica gigas extract.

10. The method according to claim 6,
wherein the (R)-7-hydroxy-8,8-dimethyl-7,8-dihydropyrano[3,2-g]chromen-2(6H)-one is prepared by:
a step (a-1) of reacting, in an organic solvent, (S)-7-hydroxy-8,8-dimethyl-7,8-dihydropyrano[3,2-g]chromen-2(6H)-one with triphenylphosphine at 45° C. to 80° C. for 30 minutes to 5 hours;
a step (a-2) of adding a compound prepared according to the step (a-1) and (R,R)-Jacobsen's catalyst to a mixed solution of NaOCl and NaHPO₄ adjusted to pH 10 to 12, and reacting at −5° C. to 10° C. for 24 to 100 hours; and
a step (a-3) of dissolving a compound prepared according to the step (a-2) and NaCNBH₃ in an organic solvent, cooling to −5° C. to 10° C., and reacting by adding BF₄OEt₂.

11. The method according to claim 6,
wherein the (6R,7S)-6-amino-7-hydroxy-8,8-dimethyl-7,8-dihydropyrano[3,2-g]chromen-2(6H)-one is prepared by:
a step (b-1) of reacting, in an organic solvent, (S)-7-hydroxy-8,8-dimethyl-7,8-dihydropyrano[3,2-g]chromen-2(6H)-one with triphenylphosphine at 40° C. to 80° C. for 30 minutes to 5 hours;
a step (b-2) of adding a compound prepared according to the step (b-1) and (S,S)-Jacobsen's catalyst to a mixed solution of NaOCl and NaHPO₄ adjusted to pH 10 to 12, and reacting at −5° C. to 10° C. for 24 to 100 hours; and
a step (b-3) of reacting a compound prepared according to the step (b-2), in a lower alcohol solvent, with ammonia for 3 to 24 hours.

12. A pharmaceutical composition for preventing or treating non-small cell lung cancer (NSCLC), comprising, as an active ingredient, a compound represented by Formula 1 below, a tautomer thereof, a stereoisomer or a mixture thereof, or a pharmaceutically acceptable salt thereof: 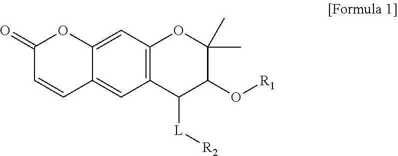
wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

13. A composition for combination therapy for preventing or treating NSCLC, comprising, as active ingredients, a compound represented by Formula 1 below, a tautomer thereof, a stereoisomer or a mixture thereof, or a pharmaceutically acceptable salt thereof, and gefitinib: 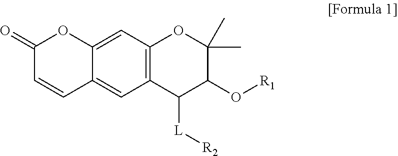
wherein in Formula 1 above,
L is a single bond or —NH—; and
R₁ and R₂ are each independently hydrogen, C₆₋₁₀ aryl-C₁₋₄ straight chain or branched chain alkyl carbonyl, C₆₋₁₀ aryl-C₁₋₄ straight chain or branched chain alkenyl carbonyl, 5-10 membered heteroaryl-C₁₋₄ straight chain or branched chain alkyl carbonyl, 5-10 membered heterocyclyl-C₁₋₄ straight chain or branched chain alkyl carbonyl, 5-10 membered heteroaryl-C₁₋₄ straight chain or branched chain alkenyl carbonyl, or 5-10 membered heterocyclyl-C₁₋₄ straight chain or branched chain alkenyl carbonyl,
wherein the aryl, heteroaryl, or heterocyclyl is unsubstituted or substituted with C₁₋₄ haloalkyl, di(C₁₋₄ alkyl)amino, or di(C₁₋₄ alkyl)amino sulfonyl.

14. A method for treating non-small cell lung cancer (NSCLC), comprising administering the pharmaceutical composition of claim 12 to a subject in need.

15. A method for treating non-small cell lung cancer (NSCLC), comprising administering the composition for combination therapy of claim 13 to a subject in need.
